(19) United States
(12) Patent Application Publication
(10) Pub. No.: US 2016/0369286 A1
(43) Pub. Date: Dec. 22, 2016

(54) COMPOSITIONS AND METHODS FOR GALLS FL AND GALLS CT MEDIATED TRANSFORMATION OF PLANTS

(71) Applicant: OREGON STATE UNIVERSITY, Corvallis, OR (US)

(72) Inventors: Lloyd W. REAM, Corvallis, OR (US); Maciej B. MASELKO, Corvallis, OR (US)

(73) Assignee: Oregon State University, Corvallis, OR (US)

(21) Appl. No.: 15/038,709

(22) PCT Filed: Nov. 21, 2014
(86) PCT No.: PCT/US2014/066913

§ 371 Date: May 23, 2016

Related U.S. Application Data
(60) Provisional application No. 61/908,079, filed on Nov. 23, 2013.

(51) Int. Cl. C12N 15/82

(52) U.S. Cl. CPC C12N 15/8205

(57) ABSTRACT

The present disclosure is directed to compositions and related methods that incorporate GALLS full-length (FL) or CT domain proteins to enhance efficiency of genetic manipulation of plants. In one aspect, the disclosure provides a modified Agrobacterium cell that comprises a first nucleic acid and a second nucleic acid that encodes a GALLS-FL protein. In another aspect, the disclosure provides a method of enhancing the single copy insertion of a first nucleic acid sequence into a plant cell genome. In another aspect, the disclosure provides a method of inducing plant susceptibility to Agrobacterium-mediated transformation, comprising providing GALLS-CT polypeptide in the cytosol of at least one cell of the plant. In another aspect, the disclosure provides a transgenic plant that comprises a heterologous nucleic acid sequence encoding GALLS-CT operably linked to a promoter sequence.

### CROSS-REFERENCE(S) TO RELATED APPLICATION(S)

This application claims the benefit of Provisional Application No. 61/908,079, filed Nov. 23, 2013, which is expressly incorporated herein by reference in its entirety.

### STATEMENT OF GOVERNMENT LICENSE RIGHTS

This invention was made with Government support under MCB-1049806 awarded by the National Science Foundation and 2012-67012-19909 awarded by the United States Department of Agriculture National Institute of Food and Agriculture. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 52834 ST25.txt. The text file is 45 KB; was created on Nov. 21, 2014; and is being submitted via EFS-Web with the filing of the specification.

### BACKGROUND

Genetic manipulation of plants has resulted in great progress towards understanding plant biology and the generation of crops with improved characteristics. Thus, genetic engineering in plants is an indispensable tool for confronting many challenges facing modern agriculture. Efficient genetic engineering is important for the development of novel plant varieties that can address nutritional deficiencies, improve disease and pest resistance, enhance or maintain growth characteristics in changing environmental conditions, and provide for production of valuable fibers, pharmaceuticals, or renewable bio-fuels. A clear understanding of the mechanisms to generate these engineered plants is also critical to avoid undesirable effects and allay public concerns.

A common approach to genetically modify a target plant involves the use of Agrobacterium tumefaciens, a gram-negative bacterium that is present in soil. Agrobacterium species, such as A. tumefaciens and the related A. rhizogenes, are plant pathogens that cause crown-gall disease and hairy root disease, respectively, in plants. These bacterial pathogens rely on horizontal gene transfer to plants to cause abnormal growth in the infected tissue. The plant tissue growth results from the transfer and expression of segments of bacterial DNA (T-DNA) from a bacterial plasmid (“tumor inducing” or “Ti-plasmid” for A. tumefaciens and “root inducing” or “Ri-plasmid” for A. rhizogenes). The T-DNA typically encodes various biosynthetic enzymes for the production of plant hormones and unusual metabolites derived from amino acids and sugars (e.g., opines), which provide the Agrobacterium a selective advantage for growth.

The T-DNA is transferred to the plant nucleus with the aid of various Agrobacterium virulence (Vir) proteins that are also encoded in the Ti- or Ri-plasmids. The Vir proteins perform various functions that facilitate the transfer and integration of bacterial genes into the plant genome. For example, in A. tumefaciens and A. rhizogenes, the T-DNA is delimited in the Ti- or Ri-plasmid by border sequences that are nicked by VirD1 and VirD2. VirD2 attaches to the 5′ end of the nicked strand. VirD2 contains a secretion signal and is transported into plant cells along with the covalently attached single-stranded T-DNA (“T-strand”). This transport requires a type IV secretion system (T4SS) that includes eleven virB-encoded proteins and VirD4. A nuclear localization sequence (NLS) in VirD2 interacts with host importin α-proteins, which mediate nuclear import.

The single-stranded DNA-binding protein (SSB) VirE2 and its chaperone VirE1 are also critical for horizontal gene transfer and, thus, pathogenesis by A. tumefaciens. Inside the plant cells, multiple VirE2 proteins attach to (or “coat”) the T-strand/VirD2 complex to form a “T-complex”. The VirE2 protects the T-strand within the T-complex from host nuclease attack and may promote the nuclear import of the T-strands. The presences of VirE2 is required only in plant cells, as demonstrated by studies where transgenic plants producing VirE2 are fully susceptible to mutant A. tumefaciens lacking virE2. VirE2-dependent gene transfer requires proteins that facilitate nuclear import of VirE2-bound T-strands, association of coated T-strands with host chromatin, and subsequent removal of VirE2 prior to T-DNA integration into the host genome. Bacterial proteins translocated into plant cells can replace some host proteins involved in these processes. For example, Arabidopsis thaliana VirE2-interacting protein 1 (VIP1) is likely to facilitate nuclear import of VirE2. VirE3, a bacterial protein that is translocated into plant cells, may replace VIP1 in plant species with limiting amounts of VIP1. Both VIP1 and VirE2 are required for association of the T-complex with host nucleosomes in vitro. Prior to T-DNA integration, VirE2 and VIP1 are removed from T-strands by VirF, a bacterial F-box protein that is translocated to plant cells. A. thaliana VIP1-binding F-box protein (VBF) can replace VirF. Both VirF and VBF target VIP1 and VirE2 for proteasomal degradation via the SCF ubiquitin pathway. A. thaliana VirE2-interacting protein (VIP2) promotes T-DNA integration by stimulating histone genes and possibly other genes important for T-DNA integration.

Many current plant transformation methods use the mechanisms involved in the horizontal gene transfer by A. tumefaciens. For example, wild-type A. tumefaciens has been modified by eliminating oncogenes that result in abnormal tissue growth, while retaining virulence (vir) genes needed to transfer T-DNA to plants. In such systems, the T-DNA of the Ti plasmid is modified to include any gene of choice to serve as the transgene for expression in the host plant. Alternatively, a “binary” plasmid can be introduced that contains the T-DNA. The binary plasmid is capable of replication within the Agrobacterium and is compatible with the mutated Ti plasmid in the cell. However, such transformation strategies, referred to herein as “VirE2-mediated transformation” strategies, have serious limitations. Unintended gene duplications, genomic rearrangements, and the absence of efficient gene targeting complicate introduction of desirable transgenes. T-DNA frequently integrates into the plant host genome in direct or inverted tandem repeats, with scrambled filler sequences at the T-DNA/plant DNA borders. Although T-DNA junctions at the right border sequence are usually precise, T-DNAs may be truncated or carry additional Ti plasmid DNA beyond the left border. Once integrated, the T-DNA structure can remain a stable and functional genetic element in the plant cell genome. However, much larger chromosomal rearrangements in the infected host genome, such as translocations, are associated with T-DNA integration events. For example, chromosomal translocations exist in 19% of 64 A. thaliana mutant lines screened from the Salk T-DNA mutant collection. Thus, the presence of multiple T-DNA copies and gross chromosomal rearrangements present a challenge for plant research. Moreover, many plant species, such as soybeans, remain relatively refractory to transformation by these and other approaches for genetic modification.

Accordingly, in spite of the advances in the field in developing techniques for genetic modification of plants, a need remains for the efficient transformation of desired plant species that avoids deleterious effects associated with gene duplications, multiple insertion events, and chromosomal rearrangements. The present disclosure addresses these needs and provides additional related benefits.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure is based on the unexpected finding that a VirE2-independent transformation pathway in A. rhizogenes, which is based on the

GALLS protein, can be co-opted to enhance the single-copy insertion of transgenes into host plant cell genomes. Accordingly, in one aspect, the present disclosure provides a modified Agrobacterium cell. The modified Agrobacterium cell comprises a first nucleic acid sequence that is heterologous to the Agrobacterium cell and a second nucleic acid sequence that encodes a GALLS-FL protein. The first nucleic acid sequence is operably linked to a first promoter sequence that facilitates or permits expression of the first nucleic acid sequence in a plant cell.

In one embodiment, the second nucleic acid sequence, which encodes the GALLS-FL protein, is operably linked to a second promoter sequence to facilitate expression of the GALLS-FL protein in the Agrobacterium cell.

In one embodiment, the GALLS-FL protein comprises a first ATP-binding domain, a second ATP-binding domain, a helicase domain, one or more TraA-like motif domains (such as 1, 2, 3, 4, or 5 TraA-like domains that are the same or different), a nuclear localization domain, and a GALLS-CT domain. In one embodiment, the GALLS-CT domain comprises at least two GALLS domains and a type-IV secretion signal. In one embodiment, the GALLS-CT domain comprises three GALLS domains and a type-IV secretion signal.

In one embodiment, the GALLS-FL protein comprises an amino acid sequence with at least 70% identity to the amino acid sequence set forth in SEQ ID NO:2. In one embodiment, the second nucleic acid sequence that encodes the GALLS-FL protein is derived from Agrobacterium rhizogenes. In one embodiment, the second nucleic acid sequence that encodes the GALLS-FL protein is heterologous to the Agrobacterium cell. In one embodiment, the modified Agrobacterium cell further comprises one or more nucleic acid sequences that encode one or more of VirA, VirG, VirB1-VirB11, VirD 1, VirD2, VirD4, VirD5, VirC1, VirC2, and VirE3. In one embodiment, the modified Agrobacterium cell does not express VirE2 polypeptide or VirEl polypeptide. In one embodiment, the modified Agrobacterium cell is Agrobacterium rhizogenes, Agrobacterium tumefaciens, or is derived therefrom.

In one embodiment, the first promoter sequence is an inducible promoter sequence. In one embodiment, the first promoter sequence is a constitutive promoter in the plant cell nucleus. In one embodiment, the first promoter sequence is a plant tissue-specific promoter. In one embodiment, the first promoter sequence is homologous to a promoter sequence endogenous to the plant cell genome.

In one embodiment, the plant cell is selected from soybean, canola, corn, cotton, rice, alfalfa, wheat, potato, tomato, pepper, and the like.

In one embodiment, the first nucleic acid sequence is in a T-DNA domain. In one embodiment, the T-DNA domain is located on a chromosome of the Agrobacterium cell. In one embodiment, the T-DNA domain is located on a plasmid in the Agrobacterium cell. In some embodiments, the plasmid can be a Ti plasmid, an Ri plasmid, or a binary plasmid.

In one embodiment, the Agrobacterium cell further comprises a third nucleic acid sequence that encodes a selectable marker. In one embodiment, the third nucleic acid sequence that encodes a selectable marker is in the same molecule as the first nucleic acid sequence. In one embodiment, the T-DNA domain comprising the first nucleic acid sequence further comprises the third nucleic acid that encodes a selectable marker.

In one embodiment, the first nucleic acid sequence and the operably linked first promoter sequence are flanked on each side by one or more T-DNA border sequences. In one embodiment, the first nucleic acid sequence and the operably linked first promoter sequence are further flanked on one side by an overdrive sequence. In one embodiment, the first nucleic acid sequence, the operably linked first promoter sequence, and the third nucleic acid sequence are flanked on each side by one or more T-DNA border sequences. In one embodiment, the first nucleic acid sequence, the operably linked first promoter sequence, and the third nucleic acid sequence are further flanked on one side by an overdrive sequence.

In another aspect, the disclosure provides a method of transforming a plant cell with a first nucleic acid sequence. The method comprises contacting the plant cell with a modified Agrobacterium cell as described herein. In one embodiment, the plant cell is stably transformed with the first nucleic acid.

In another aspect, the disclosure provides a method of enhancing a single copy insertion of a first nucleic acid sequence into a plant cell genome. The method comprises contacting the plant cell with a modified Agrobacterium cell as described herein.

In one embodiment of either of the above method aspects, the first nucleic acid sequence is heterologous to the plant cell genome.

In one embodiment of either of the above method aspects, the method comprises propagating the plant cell.

In one embodiment of either of the above method aspects, the method further comprises inducing the expression of the first nucleic acid sequence in the plant cell or progeny thereof.

In another aspect, the disclosure provides a method of inducing plant susceptibility to Agrobacterium-mediated transformation, comprising providing GALLS-CT polypeptide in the cytosol of at least one cell of the plant.

In one embodiment, the step of providing GALLS-CT polypeptide in the cytosol comprises contacting the plant cell with an Agrobacterium cell that expresses GALLS-CT polypeptide. In one embodiment, the step of providing GALLS-CT polypeptide in the cytosol comprises providing for the expression of a heterologous nucleic acid that encodes GALLS-CT in the plant cell.

In one embodiment, the heterologous nucleic acid is stably integrated into the genome of the plant cell. In another embodiment, the heterologous nucleic acid is transiently expressed in the plant cell.

In one embodiment, the step of providing GALLS-CT polypeptide in the cytosol comprises contacting the plant cell with an Agrobacterium cell that expresses GALLS-CT polypeptide and providing for the expression of a heterologous nucleic acid that encodes GALLS-CT in the plant cell.

In one embodiment, the GALLS-CT polypeptide comprises at least two GALLS domains and a type-IV secretion domain.

In one embodiment, GALLS-CT protein is encoded by a nucleic acid derived from Agrobacterium rhizogenes. In one embodiment, the GALLS-CT polypeptide has an amino acid sequence with at least 70% identity to the amino acid sequence set forth in SEQ ID NO:4. In one embodiment, the Agrobacterium-mediated transformation is mediated by the Agrobacterium GALLS pathway or Agrobacterium VirE2 pathway. In one embodiment, the plant is selected from soybean, canola, corn, cotton, rice, alfalfa, wheat, potato, tomato, pepper, and the like.

In one embodiment, the method further comprises contacting the plant with an Agrobacterium cell. In one embodiment, the Agrobacterium cell comprises a transgene capable of expression in the plant. In one embodiment, the Agrobacterium cell comprises a functional VirE2 or GALLS pathway for transformation.

In another aspect, the disclosure provides a method of enhancing the efficiency of Agrobacterium-mediated transformation in a plant. The method comprises providing GALLS-CT polypeptide in the cytosol of at least one cell of the plant. The method also comprises contacting the plant with an Agrobacterium cell comprising a transgene capable of expression in the plant cell.

In one embodiment, the step of providing GALLS-CT polypeptide in the cytosol comprises contacting the plant cell with an Agrobacterium cell that expresses GALLS-CT polypeptide. In one embodiment, the step of providing GALLS-CT polypeptide in the cytosol comprises providing for the expression of a heterologous nucleic acid that encodes GALLS-CT in the plant cell.

In one embodiment, the heterologous nucleic acid is stably integrated into the genome of the plant cell. In another embodiment, the heterologous nucleic acid is transiently expressed in the plant cell.

In one embodiment, the step of providing GALLS-CT polypeptide in the cytosol comprises contacting the plant cell with an Agrobacterium cell that expresses GALLS-CT polypeptide and providing for the expression of a heterologous nucleic acid that encodes

GALLS-CT in the plant cell.

In one embodiment, GALLS-CT polypeptide is provided in the cytosol concurrently with or prior to contacting the plant with an Agrobacterium cell.

In one embodiment, the GALLS-CT polypeptide comprises at least two GALLS domains and a type-IV secretion domain.

In one embodiment, GALLS-CT protein is encoded by a nucleic acid derived from Agrobacterium rhizogenes. In one embodiment, the GALLS-CT polypeptide has an amino acid sequence with at least 70% identity to the amino acid sequence set forth in SEQ ID NO:4. In one embodiment, the Agrobacterium-mediated transformation is mediated by the Agrobacterium GALLS pathway or Agrobacterium VirE2 pathway. In one embodiment, the plant is selected from soybean, canola, corn, cotton, rice, alfalfa, wheat, potato, tomato, pepper, and the like.

In another aspect, the disclosure provides a transgenic plant, or component thereof, comprising a cell with a heterologous nucleic acid sequence encoding GALLS-CT operably linked to a promoter sequence. In one embodiment, the heterologous nucleic acid sequence is stably integrated into the genome of the cell. In another embodiment, the heterologous nucleic acid sequence is transiently transformed into a cell.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a cartoon illustration of the structure of the GALLS-FL polypeptide and GALLS-CT polypeptide;

FIG. 2 is a Southern blot illustrating the number of transgene inserts observed in transgenic plants transformed using Agrobacterium cells with intact VirE2 or GALLS pathways; and

FIG. 3 is a graphical illustration of the enhanced transformation efficiency of Agrobacterium cells in the presence of GALLS-CT protein, whether using VirE2 or GALLS pathways.

### DETAILED DESCRIPTION

The present disclosure is based on the unexpected finding that a VirE2-independent transformation pathway in A. rhizogenes, which is based on the GALLS protein, can be co-opted to enhance the single-copy insertion of transgenes into host plant cell genomes. Furthermore, the GALLS gene produces a C-terminal fragment (“GALLS-CT”) due to an in-frame start codon. The GALLS-CT protein was found to unexpectedly enhance transformation efficiencies of Agrobacterium cells, whether based on VirE2-dependent or VirE2-independent (e.g., GALLS-dependent) pathways.

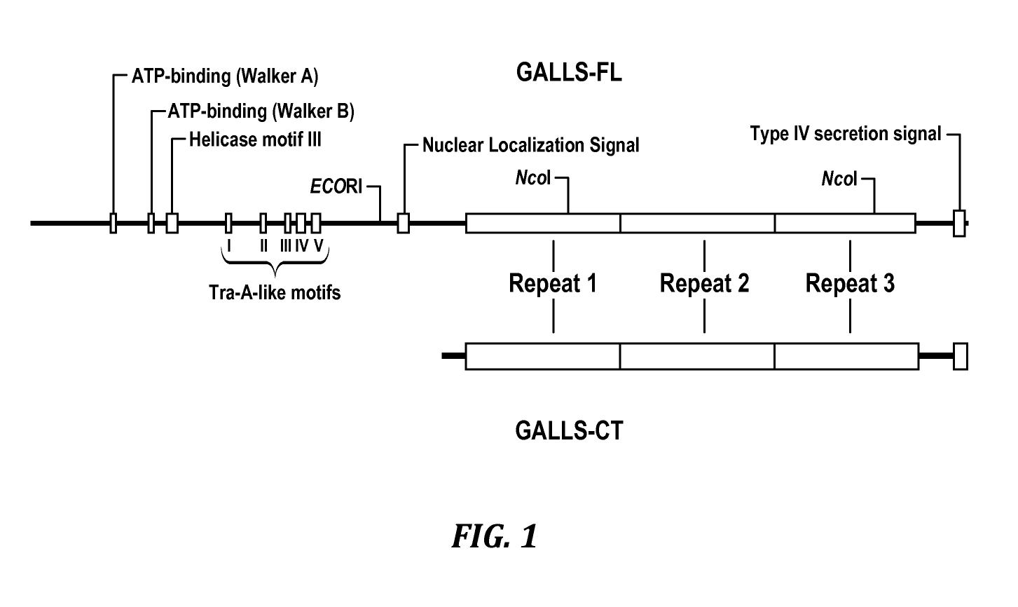

Root-inducing (Ri) plasmids of A. rhizogenes and tumor-inducing (Ti) plasmids of A. tumefaciens share many similarities, including nearly identical organization of the vir operons. One exception is that the Ri plasmid (and indeed the entire genome) of some strains of A. rhizogenes lack virE1 and virE2. For example, A. rhizogenes strain 1724 lacks virE1 and virE2 but still transfers T-DNA efficiently due to a translocated effector protein (GALLS or GALLS-FL (for “full length”)) that provides an alternative means for nuclear import of ssDNA. See Hodges, L. D., et al., “Agrobacterium rhizogenes GALLS Protein Contains Domains for ATP Binding Nuclear Localization, and Type IV Secretion,” J. Bacteriol., 188(23):8222 (2006), incorporated herein by reference in its entirety, which describes that the GALLS gene from pRi1724 restores virulence to a knockout virE2 mutation in A. tumefaciens. Furthermore, Hodges, L. D., et al., “The Agrobacterium rhizogenes GALLS Gene Encodes Two Secreted Proteins Required for Genetic Transformation of Plants,” J. Bacteriol. 191(1):355-364 (2009), incorporated herein by reference in its entirety, describes that the GALLS gene encodes two proteins: a longer 1,769 amino acid protein (GALLS, or GALLS-FL) and a truncated C-terminal domain with 962 amino acids (GALLS-CT) that is translated from an alternative in-frame start codon (methionine 808). Both GALLS proteins contain a secretion signal at their C-termini (FIG. 1) and are secreted to plant cells during infection. Although the GALLS proteins lack obvious structural similarities to VirE2, GALLS-FL fully replaces VirE2 functionality in some hosts (Kalanchoe daigremontiana), but separately expressed GALLS-CT is also required for full virulence on others (carrot and A. thaliana).

The closest relatives of GALLS-FL are helicases and proteins involved in plasmid conjugation. The N-terminus of GALLS-FL resembles the helicase/strand transferase domains of plasmid-encoded TraA (strand transferase) proteins from A. tumefaciens. This portion of GALLS-FL contains two ATP-binding motifs and a third motif found in members of a helicase-replicase superfamily (FIG. 1), which are lacking in VirE2. Mutations in any of these motifs abolish the ability of GALLS-FL to substitute for VirE2 but do not destabilize the protein. Hodges, L. D., et al., J. Bacteriol. (2009). The N-terminal region of GALLS-FL also contains five highly conserved TraA-like motifs (FIG. 1). TraA is related to TraI from the F plasmid of Escherichia coli, with helicase I activity and the ability to nick within the F origin of transfer (oriT) sequence. These helicase motifs are required for GALLS-FL to replace VirE2. Hodges, L. D., et al., J. Bacteriol. (2009).

As indicated above, GALLS-FL contains a Nuclear Localization Signal (NLS) sequence (FIG. 1) that is important to complement virE2 mutations. Deletion of the NLS severely reduces tumorigenesis, but, again, stability of the protein is not affected. A NLS from tobacco etch virus (TEV) substitutes for the native NLS, even though their lengths and amino acid sequences differ significantly (FIG. 1) showing that substantial changes to this region do not disrupt other functional domains. Hodges, L. D., et al., J. Bacteriol. (2009).

The present disclosure is based on the surprising discovery that Agrobacterium cells modified to express GALLS protein, in the absence of VirE1 and VirE2, unexpectedly results in a significantly enhanced frequency of the single-copy insertion of transgenes into the host plant cell genome. Furthermore, after further characterization, it was unexpectedly found that the GALLS-CT protein further enhances the efficiency of VirE2-mediated transformation of plants as well as GALLS-mediated transformation of plants. These results thus provide compositions, systems, and related methods for improved genetic transformation of plants. It will be apparent to persons of ordinary skill in the art that the compositions, systems, and related methods of the present disclosure can be applied to genetically modify plants without limitation to the identity of the plant, and will be especially useful in facilitating genetic modifications to species that have heretofore been recalcitrant to transformation.

In accordance with the above, the present disclosure provides a modified Agrobacterium cell. In one embodiment, the modified Agrobacterium cell comprises a first nucleic acid sequence that is heterologous to the Agrobacterium cell and a second nucleic acid sequence that encodes a GALLS-FL protein. In one embodiment, the first nucleic acid sequence is operably linked to a first promoter sequence that facilitates expression of the first nucleic acid sequence in a plant cell nucleus.

The first and second nucleic acid sequences can independently reside in the bacterial chromosomal DNA or in plasmid DNA. In one embodiment, the first and second nucleic acid sequences reside in plasmid DNA, which can be the same plasmid or different plasmids. In one embodiment, the first and second nucleic acid sequences reside in the same plasmid.

The first nucleic acid sequence can be any sequence of interest. However, the first nucleic acid sequence preferably does not appear in the genome of the wild-type Agrobacterium cell. For instance, the first nucleic acid can be any sequence that is desired to be transgenically expressed in a target plant cell. Thus, the first nucleic acid can encode any protein that confers a beneficial characteristic on a plant, such as characteristics related to disease and/or pest resistance, improved growth rate and/or resistance to adverse environmental conditions, improved food and/or seed production, improved biofuel production, and the like. In this regard, the present inventors discovered that use of the GALLS-based pathway in an Agrobacterium cell promotes an enhanced rate of single-copy insertion of a transgene into a host plant cell genome to produce a transgenic plant. Such improvement confers various advantages, such as the decreased likelihood of genomic recombination, duplication, transgene silencing, or interruption of endogenous genes. Accordingly, the first nucleic acid of this aspect of the disclosure can serve as the intended transgene for potential insertion in a host plant cell genome. Such methods are provided in greater detail below.

In one embodiment, the second nucleic acid sequence that encodes the GALLS-FL protein is operably linked to a second promoter sequence to facilitate expression of the GALLS-FL protein in the Agrobacterium cell. As used herein, the term “operably linked” refers to a juxtaposition wherein the components so described are in a relationship permitting them to function in their intended manner. Thus, the second promoter “operably linked” to the second nucleic acid sequence is disposed in the same nucleic acid molecule in such a manner that it facilitates the transcription and, thus, expression of the second nucleic acid in the intended cellular context (i.e., with the appropriate transcription factors, and the like). In one embodiment, the second promoter can be an endogenous promoter sequence for the GALLS gene in an Agrobacterium rhizogenes Ri-plasmid.

As described above, a wild-type GALLS gene from A. rhizogenes was previously characterized as encoding a longer 1,769 amino acid protein, referred to as GALLS, or GALLS-FL for “full length”, as well as a truncated C-terminal domain with 962 amino acids (GALLS-CT) that is translated from an alternative in-frame start codon (methionine 808). The nucleic acid for the reported GALLS gene is set forth in SEQ ID NO:1, and the predicted amino acid sequence of the GALLS-FL is set forth herein as SEQ ID NO:2. The nucleic acid encoding only the GALLS-CT domain is set forth herein as SEQ ID NO:3, and the predicted amino acid sequence of the GALLS-CT is set forth herein as SEQ ID NO:4. However, it is noted that the terms “FL” and “full length,” as used in reference to a particular GALLS protein, do not necessarily strictly imply or require the entire length of the predicted protein set forth in SEQ ID NO:2 or a homolog or variant thereof. Various sequence modifications and degrees of sequence identities from the reference SEQ ID NO:2 sequence are contemplated, as described in more detail below. These can include sequences with truncations and/or deletions and still be encompassed by the terms “GALLS-FL” and “GALLS full length”. In this context, the designations of “FL” and “full length” are used to merely distinguish this genus of longer GALLS proteins from the shorter GALLS-CT proteins, which correspond to a C-terminal domain encoded by the GALLS gene. Thus, “GALLS-FL” or “GALLS full length” proteins contain one or more additional identifiable domains compared to the GALLS-CT proteins, as described in more detail below.

In one embodiment, the GALLS-FL protein comprises an amino acid sequence with at least 60%, 65%, 70%, 75%, 80%, 85%, 90%, 95%, 99% identity, or any range of identity derivable therein, to the amino acid set forth in SEQ ID NO:2. In one embodiment, the GALLS-FL protein comprises the amino acid sequence of SEQ ID NO:2.

As used herein, an “amino acid” refers to any of the 20 naturally occurring amino acids found in proteins, D-stereoisomers of the naturally occurring amino acids (e.g., D-threonine), unnatural amino acids, and chemically modified amino acids. Each of these types of amino acids is not mutually exclusive. α-Amino acids comprise a carbon atom to which is bonded an amino group, a carboxyl group, a hydrogen atom, and a distinctive group referred to as a “side chain.” The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine).

The following abbreviations are used for the 20 naturally occurring amino acids: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, the term “percent identity” or “percent identical”, refers to the percentage of amino acid residues in a polypeptide sequence (or nucleotides in a nucleic acid sequence) that are identical with the amino acid sequence (or nucleic acid sequence) of a specified molecule, after aligning the sequences to achieve the maximum percent identify. Alignments can include the introduction of gaps in the sequences to be aligned to maximize the percent identity.

Several methods exist for determining percent identity and, thus, one may determine percent identity in any technologically acceptable manner. For example, a target nucleic acid or amino acid sequence can be compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from the U.S. Government's National Center for Biotechnology Information web site (world wide web at ncbi.nlm.nih.gov). Instructions explaining how to use the Bl2seq program can be found in the readme file accompanying BLASTZ. Another illustrative program that can be used for sequence alignment is Vector NTI Advance™ 9.0. Another way of calculating identity can be performed by published algorithms. Optimal alignment of sequences for comparison may be conducted by the local identity algorithm of Smith and Waterman, Adv. Appl. Math 2:482 (1981), by the identity alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The GALLS-FL protein can have any sequence modification or difference as compared to the reference SEQ ID NO:2 sequence so long as the protein retains the function to facilitate delivery of intact T-DNA to the plant cell. In this sense, the GALLS-FL, or “full length”, protein need not contain the entire length of SEQ ID NO:2, as described above. The function can be confirmed by any appropriate assay, such as described in more detail herein. Sequence variation can include conservative mutations or substitutions from the reference sequence of SEQ ID NO:2 such that it minimally disrupts the higher-level structure or the biochemical properties of the protein. Non-limiting examples of mutations that are introduced to substitute conservative amino acid residues include: positively-charged residues (e.g., H, K, and R) substituted with positively-charged residues; negatively-charged residues (e.g., D and E) substituted with negatively-charged residues; neutral polar residues (e.g., C, G, N, Q, S, T, and Y) substituted with neutral polar residues; and neutral non-polar residues (e.g., A, F, I, L, M, P, V, and W) substituted with neutral non-polar residues. Conservative substitutions can be made in accordance with the following Table 1. Nonconservative substitutions can be made as well (e.g., proline for glycine).

TABLE 1
Exemplary Amino Acid Substitutions

| Amino Acid | Substitutions |
|---|---|
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

It is noted that the GALLS-FL protein was characterized in Hodges, L. D., et al., J. Bacteriol. (2006) and Hodges, L. D., et al., J. Bacteriol. (2009), each incorporated herein by reference in their entireties. As described, various functional domains of the GALLS-FL protein were characterized and mutational studies identified regions that ablated the functionality of the protein when altered (see, FIG. 1 for a diagrammatic overview of the protein). These studies can inform the person of skill in the art as to what variation can and cannot be tolerated by the GALLS-FL, with respect to variation from the reference SEQ ID NO:2 sequence, while maintaining functionality. Accordingly, in some embodiments, the domains described below contains higher sequence identity to the corresponding amino acid sequences in SEQ ID NO:2. In some embodiments, the total sequence identity for any one or more of the domains described herein is 80%, 85%, 90%, 91%, 92%, 93%, 94%, 95%, 96%, 97%, 98%, 99% identity, or any range of identity derivable therein, to the amino acid sequence of the corresponding sequences set forth in SEQ ID NO:2. In some embodiments, one, more, or all of the domains of the GALLS-FL protein described below comprise an amino acid sequence that is the same (i.e., 100% identity) as the amino acid sequence of the corresponding domain(s) in SEQ ID NO:2.

For example, two ATP-binding domains were identified at the N-terminal region of the protein. The amino acid sequence of the first ATP-binding domain (Walker A domain) is set forth herein as SEQ ID NO:5, and corresponds to amino acids 160-173 of SEQ ID NO:2. The amino acid sequence of the second ATP-binding domain (Walker B domain) is set forth herein as SEQ ID NO:6, and corresponds to amino acids 228-240 of SEQ ID NO:2. These sequences are conserved in many proteins that bind ATP. Accordingly, in some embodiments the GALLS protein contains at least one ATP-binding domain. In some embodiments, the GALLS protein contains two ATP-binding domains. In some embodiments, the at least one or two ATP-binding domains have an amino acid sequence selected independently from the sequences set forth as SEQ ID NO:5 and SEQ ID NO:6. Moreover, the conserved lysine in the Walker A motif (position 13 of SEQ ID NO:5, or position 172 of SEQ ID NO:2) and the conserved aspartic acid in the Walker B motif (position 12 of SEQ ID NO:6, or position 239 of SEQ ID NO:2) were shown to be required for ATP binding by mutational studies. Accordingly, in some further embodiments, the GALLS-FL protein contains an amino acid residue corresponding to position 13 of SEQ ID NO:5 (or position 172 of SEQ ID NO:2) and/or position 12 of SEQ ID NO:6 (or position 239 of SEQ ID NO:2).

Additionally, a helicase motif was identified at amino acids 269-288 of SEQ ID NO:2, which is set forth herein as SEQ ID NO:7. This GALLS helicase motif was found to resemble most closely the corresponding motif in RecD helicase from Mycoplasma pulmonis (65% identical). A deletion of the amino acids 5-14 of SEQ ID NO:2, corresponding to residues 273-282 of SEQ ID NO:2, ablated function of the protein. Accordingly, in one embodiment, the GALLS-FL protein comprises an amino acid sequence corresponding to the sequence set forth in SEQ ID NO:7 or residues 269-288 of SEQ ID NO:2.

Moreover, five TraA-like motif domains were identified, set forth herein as SEQ ID NOS:8-12, and corresponding to amino acids 379-388, 446-454, 492-500, 513-527, and 541-555, respectively, of SEQ ID NO:2. Accordingly, in one embodiment, the GALLS-FL protein comprises an amino acid sequence corresponding to an amino acid sequence set forth in one of SEQ ID NOS:8-12. In one or more further embodiments, the GALLS-FL protein comprises 2, 3, 4, or 5 of the amino acid sequences corresponding to one or more amino acid sequence selected from SEQ ID NOS:8-12. In one embodiment, the GALLS-FL protein comprises amino sequences corresponding to each of the sequences set forth in SEQ ID NOS:8-12.

A first putative bipartite nuclear localization sequence (NLS) was also identified as being important for GALLS-FL function (FIG. 1). Specifically, the domains comprising amino acid residues 705-708 and 718-724 of SEQ ID NO:2, the entire continuous sequence set forth herein as SEQ ID NO:13, was identified in the GALLS-FL protein. Removal of this motif from the GALLS-FL protein severely reduced the ability of the protein to restore infectivity in a VirE2 knockout Agrobacterium. However, replacement of this domain with an unrelated NLS completely restored functionality of the protein. Thus, the specific sequence of the NLS domain is not critical. Thus, in some embodiments, the GALLS-FL protein comprises an NLS that is functional in plants. In some embodiments, the NLS comprises amino acid residues corresponding to residues 1-4 and 14-20 of SEQ ID NO:13 (or 705-708 and 718-724 of SEQ ID NO:2), with or without amino acids corresponding to some or all of the intervening residues (residues 5-13) of SEQ ID NO:13. In some embodiments, the GALLS-FL protein comprises an amino acid sequence corresponding to SEQ ID NO:13 (or 705-724 of SEQ ID NO:2).

Three GALLS repeat domains were also identified in the GALLS-FL protein. Specifically, the domains comprising amino acids 828-1093, 1117-1382, and 1406-1671 of SEQ ID NO:2, also set forth herein as SEQ ID NOS:14-16, respectively. Deletion of two of the GALLS repeat domains ablated functionality of the GALLS-FL protein. Accordingly, the GALLS-FL protein comprises at least two GALLS repeat domains. In some embodiments, the GALLS-FL protein comprises three GALLS repeat domains. In some embodiments, the two or more GALLS repeat domains comprise a sequence selected independently from SEQ ID NOS:14-16.

A type IV secretion signal was also characterized at the C-terminus of the GALLS-FL protein. Mutation studies suggested consensus secretion signal of RXX RXRXRXX (SEQ ID NO:17) for functionality, wherein X can be any amino acid residue, at the C-terminus of the GALL-FL protein. Thus, in one embodiment, the GALLS-FL protein comprises an amino acid sequence corresponding to positions 1 and 9-14 of SEQ ID NO:17, wherein the amino acids corresponding to SEQ ID NO:17 residues 1 and 9 are separated by at least 3, 4, 5, 6 or 7 amino acids. In some embodiments, the GALLS-FL protein comprises an amino acid sequence corresponding to SEQ ID NO:17. In some embodiments, the GALLS-FL protein comprises an amino acid sequence corresponding to SEQ ID NO:18, or a sequence with at least 85%, 90%, 95%, 99%, thereto, or any range derivable therein. SEQ ID NO:18 corresponds to amino acids residue positions 1751-1763 of SEQ ID NO:2.

In one embodiment, the GALLS-FL protein comprises, in order from the N-terminus to the C-terminus of the protein, a first ATP-binding domain, a second ATP-binding domain, a helicase domain, a nuclear localization domain, and a GALLS-CT domain, wherein the GALLS-CT domain comprises at least two GALLS repeat domains and a type-IV secretion signal, as described herein. In a further embodiment, the GALLS-FL protein further comprises one, two, three, four, or five TraA-like motif domains between the helicase domain and the nuclear localization domain. In one embodiment, the second nucleic acid comprises a GALLS gene. In some embodiments, the second nucleic acid sequence that encodes a GALLS-FL protein has nucleic acid sequence as set forth in SEQ ID NO:1, or a sequence with at least 60%, 65%, 70%, 75%, 80%, 85%, 90%, 95%, 99% identity thereto. In one embodiment, the second nucleic acid sequence also encodes, and can functionally express, a GALLS-CT protein. As described herein and in Hodges, L. D., et al., J. Bacteriol. (2009), the GALLS gene in A. rhizogenes encodes two proteins from the same open reading frame (designated FL and CT). The GALLS-CT specifically is translated from an in-frame internal start codon corresponding to methionine 808. GALLS-CT, and potential variation encompassed thereby is described in more detail below.

In one embodiment, the second nucleic acid sequence that encodes the GALLS-FL protein is derived from Agrobacterium rhizogenes. As used herein, the term “derived from” indicates that the nucleic acid encoding the GALLS-FL was originally obtained from an A. rhizogenes cell. The nucleic acid can comprise various mutations implemented therein and, thus, deviate from the sequence of the A. rhizogenes cell of origin.

In one embodiment, the second nucleic acid sequence that encodes the GALLS-FL protein is heterologous to the Agrobacterium cell. As used herein, the term “heterologous” indicates that the sequence, copy number, or functional association with a promoter of the nucleic acid is not naturally occurring in the cell. Thus, the second nucleic acid can comprise a sequence or be associated with a promoter sequence that differs from any naturally occurring sequence in the Agrobacterium cell. Additionally, the second nucleic acid can be heterologous by virtue of being a non-natural duplicate copy of the sequence within the Agrobacterium cell. Typically, the Agrobacterium cell is modified (i.e., engineered) through standard recombinant techniques to contain the second nucleic acid sequence.

The modified Agrobacterium cell encompassed by the present disclosure has basic biological functionality and, thus, encodes the basic proteins required for sustaining at least temporary cellular life, as are readily identifiable by persons of skill in the art. As described above, the Ti and Ri plasmids of A. tumefaciens and A. rhizogenes, respectively, have similar structures and organization of the virulence factor (vir) operons that facilitate horizontal gene transfer, with the exception that the Ri plasmids of many A. rhizogenes do not contain genes encoding VirE1 or VirE2. The remaining vir operons comprise genes encoding various factors that facilitate the processing and translocation of the T-DNA to the plant cell and nucleus. Accordingly, in one embodiment, the modified Agrobacterium cell further comprises one or more nucleic acid sequences that encode one or more of VirA, VirG, VirB1-VirB11, VirD1, VirD2, VirD4, VirD5, VirC1, VirC2, and VirE3 proteins. In further embodiments, the genome of Agrobacterium cell further comprises one or more of genes encoding any of the following: known chromosome-encoded virulence factors including ChvA, ChvB, ChvD, ChvE, ChvG, ChvI, ChvH, and the like; and housekeeping factors such as PckA, Mia, AopB, and KatA. In another embodiment, the Ri or Ti plasmid also comprises nucleic acids that encode various nonessential, but enhancing factors, such as VirD5, VirF, VirH1, VirH2, and VirJ. In some embodiments, such additional genes encode factors that increase efficiency of the modified Agrobacterium cell's ability to transform plant cells or enhance the target range of susceptible plant species.

Generally, the genes encoding these additional proteins are disposed on one or more plasmids, such as a Ti or Ri plasmid. However, it will be appreciated that such genes can also be disposed in the chromosomal DNA of the modified Agrobacterium cell. The location of the encoding genes need not be limited to the naturally occurring loci of the genes.

In one embodiment, the modified Agrobacterium cell does not express VirE2 and/or VirE1. In one embodiment, the modified Agrobacterium cell does not encode a functional VirE2 and/or VirE1 protein.

In one embodiment, the modified Agrobacterium cell is, or is derived from, Agrobacterium rhizogenes or Agrobacterium tumefaciens. In this regard, the term “derived from” refers to the parental strain of Agrobacterium, which is engineered or modified to result in the altered version encompassed by the present disclosure. The parental strain itself can be a wild-type or previously modified or engineered strain.

The first promoter encompassed by the present disclosure can be any promoter sequence that promotes or permits expression of the first nucleic acid sequence in the plant cell. Persons of ordinary skill in the art can readily identify appropriate promoters that can provide this function in the plant of interest. In one embodiment, the first promoter is inducible. For example, the promoter is capable of induction to facilitate expression of the first nucleic acid sequence after it is delivered to a plant cell by the additional administration of the appropriate transcription factors or composition to the plant cell. Many of such promoters are known in the art. For illustrative non-limiting examples, see: inducible promoters from the ACEI system that responds to copper (Mett et al., PNAS 90:4567-4571 (1993)); In2 gene from maize that responds to benzenesulfonamide herbicide safeners (Hershey et al., Mol. Gen. Genetics 227:229-237 (1991) and Gatz et al., Mol. Gen. Genetics 243:32-38 (1994)); and Tet repressor from Tn10 (Gatz et al., Mol. Gen. Genetics 227:229-237 (1991)). A particularly useful inducible promoter may be a promoter that responds to an inducing agent to which plants do not normally respond. An exemplary inducible promoter may be the inducible promoter from a steroid hormone gene, the transcriptional activity of which may be induced by a glucocorticosteroid hormone. Schena et al., Proc. Natl. Acad. Sci. U.S.A. 88:0421 (1991). Another example is an estrogen receptor-based transactivator, XVE, which mediates highly inducible gene expression in transgenic plants. Zuo, J. et al., Plant J. 24:265-273 (2000). In another example, the promoter is capable of induction to facilitate expression of the first nucleic acid by endogenous factors produced by the plant upon certain conditions.

In another embodiment, the first promoter can be a constitutive promoter. Any constitutive promoter can be used in the instant invention. Exemplary constitutive promoters include, but are not limited to: promoters from plant viruses, such as the 35S promoter from CaMV (Odell et al., Nature 313:810-812 (1985)); promoters from rice actin genes (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); and maize H3 histone (Lepetit et al., Mol. Gen. Genetics 231:276-285 (1992) and Atanassova et al., Plant Journal 2(3):291-300 (1992)). The ALS promoter, Xbal/NcoI fragment 5′ to the Brassica napus ALS3 structural gene (or a nucleotide sequence similar to said Xbal/NcoI fragment), represents a particularly useful constitutive promoter. See PCT application WO 96/30530. Another constitutive promoter is described in Lee, Y-L., et al., Plant Physiology 145:1294-1300 (2007). In some embodiments, the first promoter is capable of promoting relatively stable and long term expression of the first nucleic acid sequence in the plant cell environment. The promoter can be capable of interacting with one or more transcription factors endogenous to the host plant cell to provide for expression in the cell.

In one embodiment, the first promoter is a plant tissue-specific promoter. Thus, the first nucleic acid is capable of producing the protein product exclusively, or preferentially, in a specific tissue. Exemplary tissue-specific or tissue-preferred promoters include, but are not limited to: a root-preferred promoter—such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

In one embodiment, the first promoter is homologous to an endogenous plant promoter. Thus, in further embodiments, the first promoter can have a high sequence identity to the endogenous plant promoter, such as 60%, 70%, 80%, 90%, 95%, 99%, 100%, or any range derivable therein, to the endogenous plant promoter. Many plant promoters are well-known, and have been used for transgenic technologies in plants and, thus, can be readily applied to the present disclosure.

The present disclosure encompasses any plant cell. The plant cell can be from any agriculturally or scientifically important plant species, cultivar or type. For example, the plant cell can be from soybean, canola, corn, cotton, rice, alfalfa, wheat, potato, tomato, pepper, and the like.

In one embodiment, the first nucleic acid sequence and the operatively linked first promoter sequence are in a T-DNA domain. In one embodiment, the T-DNA domain is located on a chromosome of the modified Agrobacterium cell. In one embodiment, the T-DNA domain is located on a plasmid in the modified Agrobacterium cell. The plasmid can be a Ti (“tumor inducing”) plasmid or Ri (“root inducing”) plasmid, depending on the parental strain (i.e., strain of origin) of the modified Agrobacterium cell. Alternatively, the T-DNA domain is in a “binary” plasmid vector. Binary plasmid vectors are broad-host-range plasmids with an origin of replication compatible with the Ti plasmid. Some very useful binary vectors have an origin from the Ri plasmid, which is also compatible with the Ti plasmid. Such vectors stabilize large T-DNA insertions and can readily replicate within the Agrobacterium cell. When binary plasmids are used to provide the T-DNA, the existing Ti (or Ri, when applicable) plasmid is typically disarmed by removing the naturally occurring oncogenes, but preserving the vir genes that are required for the horizontal transfer of the transgene-containing T-DNA. An example of a binary vector is an IncP plasmid, which is well-known in the art. Another example of a useful binary vector is pCAMBIA2300. The pCAMBIA2300 binary vector has T-DNA borders flanking an nptII (kanamycin resistance) gene driven by a CaMV promoter, to provide expression in plants. The T-DNA region also contains a multiple cloning site (MCS) into which transgenes can be inserted. Other features include a high-copy origin of replication from pBR322 (i.e. ColE1), which works in E. coli, and broad-host-range ori and plasmid stability (partitioning) genes from plasmid pVS, which allow replication in Agrobacterium.

The T-DNA domain can further comprise additional features that are typically recognized as being part of the wild-type T-DNA domain. For example, the T-DNA can comprise border sequences at one and preferably both ends of the T-DNA domain. Thus, in one embodiment, the first nucleic acid sequence, the operatively linked first promoter sequence, and any additional optional sequence are together flanked on one or preferentially both sides by T-DNA border sequences. T-DNA border sequences are familiar in the art. The one or more border sequences comprise nucleotide repeat sequences, which often comprise imperfect ˜24 base direct repeats. In one embodiment, the T-DNA border sequence can be recognized by the Agrobacterium VirD1 and/or VirD2 proteins. The repeat that initiates formation of single stranded T-strand has been termed the “right border” (RB), while the repeat terminating formation of single-stranded T-DNA has been termed the “left border” (LB). Thus, in a further embodiment, the T-DNA border sequence can be recognized and nicked by the Agrobacterium VirD1 and/or VirD2 proteins.

Comparison of the RB and LB sequences from a variety of Agrobacterium strains indicated that both RB and LB share a consensus motif, which indicates that other elements may be involved in modulating the efficiency of RB processing. Cis-acting sequences next to the RB are present in many Agrobacterium strains, including A. tumefaciens and A. rhizogenes. These sequences promote wild-type virulence (Veluthambi, K., et al., J. Bacteriol. 170:1523-1532 (1988); Shurvinton, C. E., and W. Ream, J. Bacteriol. 173:5558-5563 (1991); Toro et al., J. Bacteriol., 171(12):6845-6859 (1989); Toro et al., Proc. Natl. Acad. Sci. USA, 85:8558-8562 (1988); Hansen et al., Plant Mol. Biol., 20(1):113-122 (1992)). The sequence in A. tumefaciens is often referred to as the “overdrive” or “T-DNA transmission enhancer”. See Peralta et al., EMBO J. 5(6):1137-1142 (1986). In A. rhizogenes the sequence is often referred to as the “T-DNA transfer stimulator sequence” (TSS). See Hansen et al., Plant Mol. Biol., 20(1):113-122 (1992). The overdrive (“OD”) sequence was initially defined as a particular 24-bp motif present immediately in front of the RB repeat of octopine Ti TL-DNA (Peralta et al., EMBO J. 5(6):1137-1142 (1986)). A similar sequence is present in front of the RB repeat of octopine Ti TR-DNA and also in front of nopaline Ti RB and agropine Ri TL right border (Peralta et al., EMBO J. 5(6):1137-1142 (1986), Shaw et al., Nucleic Acids Res., 12(15):6031-6041 (1984), Barker et al., Plant Mol. Biol., 2:335-350 (1983), Slightom et al., EMBO J., 4(12):3069-3077 (1985)). Further comparison of different A. tumefaciens strains revealed a 8-bp overdrive core sequence present in front of all right border sequences including nopaline strain pTiT37, octopine strain pTiA6 and A. rhizogenes pRiA4 (Peralta et al., EMBO J. 5(6):1137-1142 (1986)). Accordingly, in one embodiment, the first nucleic acid sequence, the operatively linked first promoter sequence, and any additional optional sequence are together flanked on at least one side with an overdrive sequence or TSS sequence. In one embodiment, the overdrive or TSS sequence is on the right hand border (RB) of the T-DNA.

In one embodiment, the T-DNA domain also comprises a third nucleic acid sequence that encodes a selectable marker. Often, the selectable marker is operably linked to a regulatory element (a third promoter, for example) that allows any cells that are transformed with the T-DNA to be either recovered by negative selection (i.e., inhibiting growth of cells that do not contain the selectable marker gene) or by positive selection (i.e., screening for the product encoded by the genetic marker). Many selectable marker genes for transformation are well known in the transformation arts and include, for example, genes that code for enzymes that metabolically detoxify a selective chemical agent which may be an antibiotic or an herbicide, or genes that encode an altered target which may be insensitive to the inhibitor. Positive selection methods are also known in the art.

As described herein, the present inventors made the discovery that Agrobacterium modified to express the full length GALLS gene can mediate transformation of host plant cells. Accordingly, in another aspect, the present disclosure provides a method of transforming a plant cell with a first nucleic acid sequence. The method comprises contacting the plant cell with the modified Agrobacterium described herein.

In one embodiment, the plant cell is stably transformed with the first nucleic acid (and operably linked first promoter). Alternately stated, the first nucleic acid (and operably linked first promoter) are integrated stably into the genome of the plant cell. In another embodiment, the first nucleic acid (and operably linked first promoter) are not stably integrated into the genome of the plant cell, but instead can be transiently expressed in the plant cell.

It is also described herein that the inventors made the unexpected discovery that modified Agrobacterium that express the full length GALLS gene can mediate transformation of host plant cells with a higher rate of single copy insertion of the transgene. This is beneficial because this new approach avoids problems associated with existing technologies that result in higher rates of multiple copy insertions, including higher frequency of interrupted endogenous genes and higher duplication and rearrangement rates, and less control over expression of the transgene. Thus, in one embodiment, the present method is a method of enhancing the single copy insertion of a first nucleic acid sequence into a plant cell genome and comprises contacting the plant cell with the modified Agrobacterium described herein.

In this aspect, the first nucleic acid sequence is a transgene that is intended to be expressed in the plant host cell. In one embodiment, the first nucleic acid is heterologous to the plant cell. As used herein, the term “heterologous” indicates that the nucleic acid is not naturally occurring in the plant cell genome or that the association of the nucleic acid with a particular regulatory sequence (e.g., promoter) does not naturally occur in the plant genome. Thus, the term encompasses situations where the transgene comprises a naturally occurring coding sequence from the plant operably linked to a promoter that is not naturally associated with the nucleic acid sequence in the plant, even if the promoter is also a plant-derived promoter. The term also encompasses use of a plant-derived nucleic acid sequence with mutations therein that are not naturally occurring in the plant cell.

As used herein, the term “enhancing the single copy insertion of a first nucleic acid sequence” indicates the increased likelihood that the transgene will be inserted into the plant cell genome only once. An increased likelihood refers to any increase in probability compared to a reference transformation technology, such as the use of Agrobacterium bacteria employing a VirE2-based pathway for transformation. The increase can indicate an increase of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, or any range derivable therein, of the rate of single copy insertions provided by a reference technology for transformation. Furthermore, the increase can extend beyond a 100% increase (i.e., 2-fold increase) of the rate exhibited by the reference technology, such as 2.5-fold, 3-fold, 3.5-fold, 4-fold, 4.5-fold, 5-fold, 10-fold, or more, or any range derivable therein. The reference technology can be any existing technique used to transform plants. In one embodiment, the reference technology can be an Agrobacterium-based technology. In one embodiment, the reference technology can be an Agrobacterium-based technology that employs a VirE2-based pathway for transformation. The rate can be readily established by transformation, followed by restriction digestion of the plant chromosomal DNA and Southern blot to determine the number of insertion copies, as is described in more detail below. For example, as described below in more detail, the GALLS-FL-mediated transformation technique resulted in a 55% rate of single copy insertions compared to 15% single copy insertion rate observed for VirE2-mediated technique. This represents about a 3.67-fold increase (55/15) in single copy insertion rate over the rate of the VirE2 reference technique.

In one embodiment, the method comprises propagating the cell. In a further embodiment, the method comprises first selecting one or more plant cells that have been successfully transformed to separate the one or more plant cells from unsuccessfully transformed cells prior to propagation. In this regard, as described above, the modified Agrobacterium cell can include a nucleic acid that encodes for a selectable marker. In some embodiments, the selectable marker can confer a resistance to the one or more plant cells or confer some characteristic that permits the one or more plant cells to be separated from the cells without the selectable marker. Plant cell propagation can proceed according to any of many well-known culturing techniques appropriate for the particular type of plant cell. These propagation techniques can be readily applied by persons of ordinary skill in the art and do not limit the present disclosure.

As described above, the first nucleic acid can be operatively linked to a first promoter sequence. The first promoter can be an inducible promoter. In one embodiment, the method further comprises inducing expression of the first nucleic acid in the plant cell or its progeny by administering or facilitating the correct components that interact with the first promoter to induce expression. Any known inducible promoter can be used as appropriate in plant cells. The conditions that promote induction of the linked first nucleic acids are therefore known in the art and are not expanded upon here.

The inventors have also demonstrated the unexpected finding that the presence of the C-terminal domain of GALLS protein, GALLS-CT, can enhance the transformation efficiency by Agrobacteria, regardless of whether the Agrobacteria cell uses the GALLS or VirE2 pathway for transformation (see description below). Accordingly, in another aspect, the present disclosure provides a method of inducing plant susceptibility to Agrobacterium-mediated transformation. The method comprises providing GALLS-CT polypeptide in the cytosol of at least one cell of the plant. In one embodiment, the Agrobacterium-mediated transformation is mediated by the Agrobacterium GALLS pathway. In one embodiment, the Agrobacterium-mediated transformation is mediated by the Agrobacterium VirE2 pathway.

In one embodiment, the step of “providing GALLS-CT polypeptide” comprises contacting the plant cell with an Agrobacterium cell that expresses GALLS-CT polypeptide. In another embodiment, the step of “providing GALLS-CT polypeptide” comprises providing for the expression of a heterologous nucleic acid that encodes GALLS-CT in the plant cell. In one embodiment, the heterologous nucleic acid is stably integrated into the genome of the plant cell. One observed benefit of the present method is that constitutive expression of high levels GALLS-CT in plants (i.e., in stably transformed plants) did not appear to cause disease or any reduction in fertility or growth rate (not shown). In another embodiment, the heterologous nucleic acid is transiently expressed in the plant cell. Any known technique for transgenic engineering of the target host plant can be used to provide for the expression of a heterologous nucleic acid encoding GALLS-CT in the plant. Such techniques include the compositions and methods described herein with respect to GALLS-FL mediated transformation in plants. Typically, once a transgenic plant (GALLS-CT+) is generated, the plant line would be propagated and maintained for use as the basis for further genetic transformations. In some embodiments, the step of “providing GALLS-CT polypeptide” comprises contacting the plant cell with an Agrobacterium cell that expresses GALLS-CT polypeptide and providing for the expression of a heterologous nucleic acid that encodes GALLS-CT in the plant cell, each element as described herein.

In one embodiment, the method further comprises contacting the plant with an Agrobacterium cell comprising a transgene capable of expression in the plant cell. The Agrobacterium cell can be any Agrobacterium cell that harbors the intended transgene as well as sufficient virulence infrastructure to facilitate the transfer of the transgene (in T-DNA) to the plant cell in a form that permits its expression (i.e., protected from degradation). Such virulence infrastructure is well-understood in the art and is explained in more detail above. However, the Agrobacterium cell need not express VirE1 or VirE2, but can incorporate an alternative pathway, such as the GALLS pathway, as described herein. Thus, the method of the present aspect can incorporate use of the modified Agrobacterium cell described herein with respect to initial aspects of the disclosure. In this regard, the transgene is equivalent to the first nucleic acid of the modified Agrobacterium cell as described above.

In one embodiment, the GALLS-CT polypeptide is provided in the cytosol concurrently with the step of contacting the plant with the Agrobacterium cell. For example, the Agrobacterium cell itself may express and deliver the GALLS-CT along with (or simultaneously with) the transgene. In another embodiment, the GALLS-CT polypeptide is provided in the cytosol prior to the step of contacting the plant with the Agrobacterium cell. For example, expression of a heterologous gene encoding the GALLS-CT polypeptide, whether transiently or stably transformed, can be induced in the plant cell prior to the step of contacting the plant with the Agrobacterium cell. In one embodiment, the GALLS-CT polypeptide is provided in the cytosol anytime between about 1 hour and about 48 hours, or more, prior to the step of contacting the plant with the Agrobacterium cell. For example, induction of expression in the plant of a heterologous gene encoding GALLS-CT can be performed between about 1 hour and about 48 hours prior, or more, to the step of contacting the plant with the Agrobacterium cell. In one embodiment, the GALLS-CT polypeptide is provided in the cytosol between about 12 hours prior to about 36 hours to the step of contacting the plant with the Agrobacterium cell.

As described above, the GALLS-CT protein is expressed from the GALLS gene, starting translation at an internal in-frame start codon (corresponding codon 808 of the full length gene encoding a methionine). Hodges, L. D., et al., J. Bacteriol. (2009), incorporated herein by reference in its entirety. The GALLS-CT polypeptide of the present method has at least two GALLS repeat domains. In one embodiment, the GALLS-CT polypeptide has three GALLS repeat domains. The GALLS repeat domains can comprise amino acid sequences independently selected from the sequences set forth in SEQ ID NOS:14-16, or any sequence with at least 70%, 75%, 80%, 85%, 90%, 91%, 92%, 93%, 94%, 95%, 96%, 97%, 98%, 99% identity thereto (or any range of identity derivable therein). As also described above, the GALLS-CT polypeptide has a type IV secretion signal at the C-terminus. Mutation studies suggested consensus secretion signal of RXXXXXXXRXRXRXX (SEQ ID NO:17) for optimal functionality, wherein X can be any amino acid residue, at the C-terminus of the GALLS-CT protein. Thus, in one embodiment, the GALLS-CT protein comprises an amino acid sequence corresponding to positions 1 and 9-14 of SEQ ID NO:17, wherein the amino acids corresponding to SEQ ID NO:17 residues 1 and 9 are separated by at least 3, 4, 5, 6 or 7 amino acids. In some embodiments, the GALLS-CT protein comprises an amino acid sequence corresponding to SEQ ID NO:17. In some embodiments, the GALLS-CT protein comprises an amino acid sequence corresponding to SEQ ID NO:18, or a sequence with at least 85%, 90%, 95%, 99%, thereto, or any range derivable therein.

In one embodiment, the overall GALLS-CT protein has a polypeptide sequence with at least 75%, 80%, 85%, 90%, 95%, or 99% sequence identity, or any range derivable therein, to the amino acid sequence set forth in SEQ ID NO:4. The sequence variation can encompass any mutations that do not ablate functionality. Accordingly, in some embodiments, the sequence variation from the reference SEQ ID NO:4 comprises conservative amino acid substitutions. In some embodiments, the sequence variation from the reference SEQ ID NO:4 preserve the amino acid sequence structure demonstrated in prior mutational studies, described above, to facilitate functionality of the GALLS-FL protein. In some embodiments, the GALLS-CT protein is encoded by a nucleic acid derived from an A. rhizogenes cell or strain. In one embodiment, the GALLS-CT protein has the polypeptide sequence set forth in SEQ ID NO:4. In some embodiments, the GALLS-CT protein is encoded by a nucleic acid with the sequence set forth in SEQ ID NO:3, or a sequence with at least 75%, 80%, 85%, 90%, 95%, or 99% sequence identity thereto.

This and other methods of the disclosure encompass any plant. The plant can be from any agriculturally or scientifically important plant species, cultivar or type. For example, the plant cell can be from soybean, canola, corn, cotton, rice, alfalfa, wheat, potato, tomato, pepper, and the like. A benefit conferred from the present disclosure, specifically regarding the present method, is the ability to genetically manipulate plant species that heretofore have been refractory to stable transformation efforts or are at least more difficult to transform, such as soybeans. Without being limited to any particular theory, one hypothesis is that GALLS-CT binds to one or more plant factors in the cell that are involved in signaling cascades that suppress immune responses in the plant. Thus, the plant becomes more sensitive to Agrobacterium infection. An Agrobacterium harboring a transgene of interest is, thus, more likely to transfer the T-DNA (containing the transgene of interest) to the plant cell in an intact form, even if the plant was previously refractory to such Agrobacterium-mediated transformation.

In another aspect, the present disclosure provides a method of enhancing the efficiency of Agrobacterium-mediated transformation in a plant. The method comprises providing GALLS-CT polypeptide in the cytosol of at least one cell of the plant and contacting the plant with an Agrobacterium cell comprising a transgene capable of expression in the plant cell.

The elements of this method are as described above, for example, as described in the context of the method of inducing plant susceptibility to Agrobacterium-mediated transformation, which comprises providing GALLS-CT polypeptide in the cytosol of at least one cell of the plant.

In another aspect, the present disclosure provides a transgenic plant, or component thereof, comprising a cell that with a heterologous nucleic acid encoding GALLS-CT operably linked to a promoter sequence. The GALLS-CT is described in more detail above. The heterologous nucleic acid can be stably integrated into the plant DNA or it can be separate from the plant DNA yet being capable of transient expression. The promoter sequence can be any appropriate promoter, described above, that facilitates expression of the GALLS-CT in the plant cell. The promoter can be a constitutive, inducible, and/or plant tissue specific promoter, as known in the art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, refer to this application as a whole and not to any particular portions of the application. Words such as “about” and “approximately” imply minor variation around the stated value, usually within a standard margin of error, such as within 10% or 5% of the stated value.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing aspects or embodiments can be combined or substituted for elements in other aspects or embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Unless defined otherwise, all technical and scientific terms used herein have the meaning commonly understood by one of ordinary skill in the art to which this invention belongs. For example, see Sambrook, J., and Russell, D. W., eds., Molecular Cloning: A Laboratory Manual, 3rd ed., Cold Spring Harbor Laboratory Press, Cold Spring Harbor, N.Y. (2001), and Ausubel, F. M., et al., Current Protocols in Molecular Biology (Supplement 47), John Wiley & Sons, New York (1999), which are incorporated herein by reference, for definitions and terms of the art. Any other publications cited herein, and the subject matter for which they are cited, are hereby specifically incorporated by reference in their entireties.

The following descriptions illustrate various embodiments of the present disclosure for purposes of explaining but not limiting the disclosure.

A Comparison of virE2- and GALLS-Based Transformation of Arabidopsis thaliana using Engineered Agrobacterium tumefaciens.

Arabidopsis thaliana (ecotype Col-0) were transformed by strains of A. tumefaciens capable of VirE2 or GALLS-dependent transformation. The base strain used was a disarmed A. tumefaciens strain At1872, which was derived from A. tumefaciens EHA105 by deleting the virE2 gene. The At1872 strain comprised a “binary” plasmid, pCAMBRIA2300, which contained the T-DNA, in this case a plant-expressed kanamycin resistance gene to provide a selectable marker. The strains were further differentiated by comprising the following: 1) no additional plasmid (referred to as the Atm015 strain to serve as negative control); 2) a modified a pVMC plasmid containing virE1 and virE2 expressed from the virE operon promoter (referred to as the Atm016 strain); and 3) a modified a pVMC plasmid containing the GALLS gene (see SEQ ID NO:1) with its native promoter (referred to as the Atm017 strain). The A. tumefaciens strain At564, with wild-type virE2 and pCAMBRIA2300 binary plasmid, referred to as the Atm018 strain, was used as the positive control. This system allowed for a direct comparison of GALLS- and VirE2-mediated transformation in an isogenic background. The pUCD2 plasmid is generally described in Close, T. J., et al., Plasmid. 12(2):111-118 (1984) and the pTiEHA105 plasmid is generally described in Hood, E. E., et al., Trans Res 2:208-218 (1993), each of which are incorporated by reference in their entireties.

The A. thaliana plants were transformed using the floral dip method. Seeds were tested from plants infected by each of these strains for their ability to germinate and produce plantlets on MS medium containing kanamycin. After screening >10,000 seeds pooled from three independent floral dip experiments it was determined that GALLS-mediated transformation was approximately 4-fold more efficient than VirE2-mediated transformation.

Assessment of the Transgene Copy Number in Arabidopsis thaliana Resulting from virE2- and GALLS-Based Gene Transfer from Agrobacterium tumefaciens.

Arabidopsis thaliana Col-0 flowers were inoculated with non-oncogenic strains of A. tumefaciens At1872 (derived from EHA105, described above) harboring a T-DNA (on pCAMBRIA2300) that confers kanamycin resistance to transformed plants, and a plasmid (derived from pVMC) that expresses either 1) no additional plasmid (referred to as the Atm015 strain to serve as negative control); 2) a modified a pVMC plasmid containing virE1 and virE2 expressed from the virE operon promoter (referred to as the Atm016 strain); and 3) a modified a pVMC plasmid containing the GALLS gene (see SEQ ID NO:1) with its native promoter (referred to as the Atm017 strain). The A. tumefaciens strain At564, with wild-type virE2 and pCAMBRIA2300 binary plasmid, referred to as the Atm018 strain, was used as the positive control. See above description. Thus, apart from the pVMC binary plasmids, the experimental Agrobacterium strains Atm016 and Atm017 were otherwise isogenic. Kanamycin-resistant seedlings were selected on half-strength Murashige-Skoog agar (0.7%) containing 30 ug/ml kanamycin. Genomic DNA was extracted from individual seedlings produced after exposure to either the Atm016 strain or Atm017 strain and digested with a restriction endonuclease (EcoRI) that cuts once within the T-DNA. Restriction fragments were separated by agarose gel electrophoresis, denatured with alkali, and transferred to nylon membranes by capillary blotting. Blots were probed with ³²P-labeled T-DNA, and restriction fragments containing T-DNA sequences were detected using a phosphorimager.

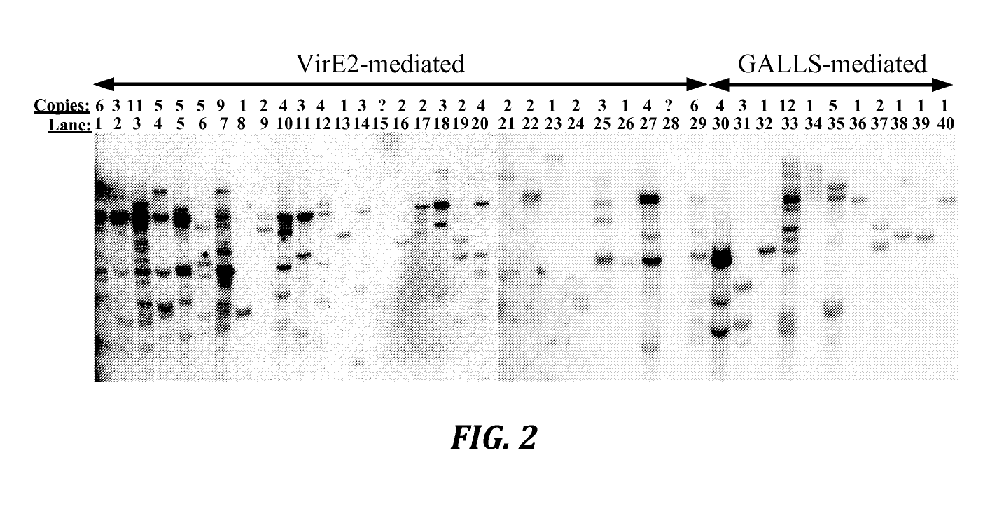

FIG. 2 illustrates the Southern blots of the restriction fragments containing the transgene insertions from each plant. Because the probe anneals to the T-DNA only on one side of the EcoRI site, each labeled band represents one copy of the T-DNA joined to plant DNA. Faint bands (e.g., the smallest band in lane 31) likely result from truncated T-DNAs, which have less overlap with the probe than do intact T-DNAs. Multiple T-DNA fragments that co-migrate may produce strong bands (e.g., the top band in lane 30) with signals that exceed those produced by intact single-copy T-DNAs. Lanes 1-29 contain DNAs from transgenic A. thaliana produced by VirE2-mediated transformation events (after exposure to the Atm016 strain), whereas lanes 30-40 contain DNAs resulting from GALLS-mediated events (after exposure to the Atm017 strain). As illustrated, there were 27 events of transgenic A. thaliana produced by VirE2-mediated transformation. Of the 27 events, four (15%) were single-copy transgene insertions. In contrast, of the 11 transgenic A. thaliana produced by GALLS-mediated transformation, six (55%) were single-copy transgene insertions.

This data demonstrates that transformation mediated with GALLS protein, independent of the VirE2 pathway, enhances efficient insertion of single transgenes in the plant host genome.

In a separate experiment, select transgenic A. thaliana produced by either VirE2-mediated transformation or GALLS-mediated transformation were further assessed to characterize the transgene insertion site. Thermal asymmetric interlaced PCR (TAIL-PCR) was performed, followed by sequencing and BLAST search was used to locate the insertion site and assess the transgene structure of the insertions, and specifically the GALLS-mediated single insertion plants. Both lines (VirE2-mediated and GALLS-mediated transgenic lines) had intact transgenes, which indicates that GALLS-dependent transformation is capable of protecting the T-DNA from nuclease attack while transiting to the cytoplasm and into the nucleus.

This data demonstrates that GALLS-mediated transformation results in transformation of the complete transgene into the host plant genome.

The Effect of the C-Terminal Portion of the GALLS Protein on the Efficiency of Agrobacterium-Mediated Plant Transformation.

It was previously described that the GALLS gene in A. rhizogenes encodes two proteins: the full length GALLS protein (also referred to as GALLS-FL) as well as a truncated C-terminal domain (GALLS-CT), which is translated from an alternative in-frame start codon (corresponding to a methionine encoded by codon 808). See Hodges, L. D., et al., “The Agrobacterium rhizogenes GALLS Gene Encodes Two Secreted Proteins Required for Genetic Transformation of Plants,” J. Bacteriol. 191(1):355-364 (2009), incorporated herein by reference in its entirety. Accordingly, a study was performed to ascertain the role or effect of the GALLS-CT protein on transformation of plants by Agrobacterium cells.

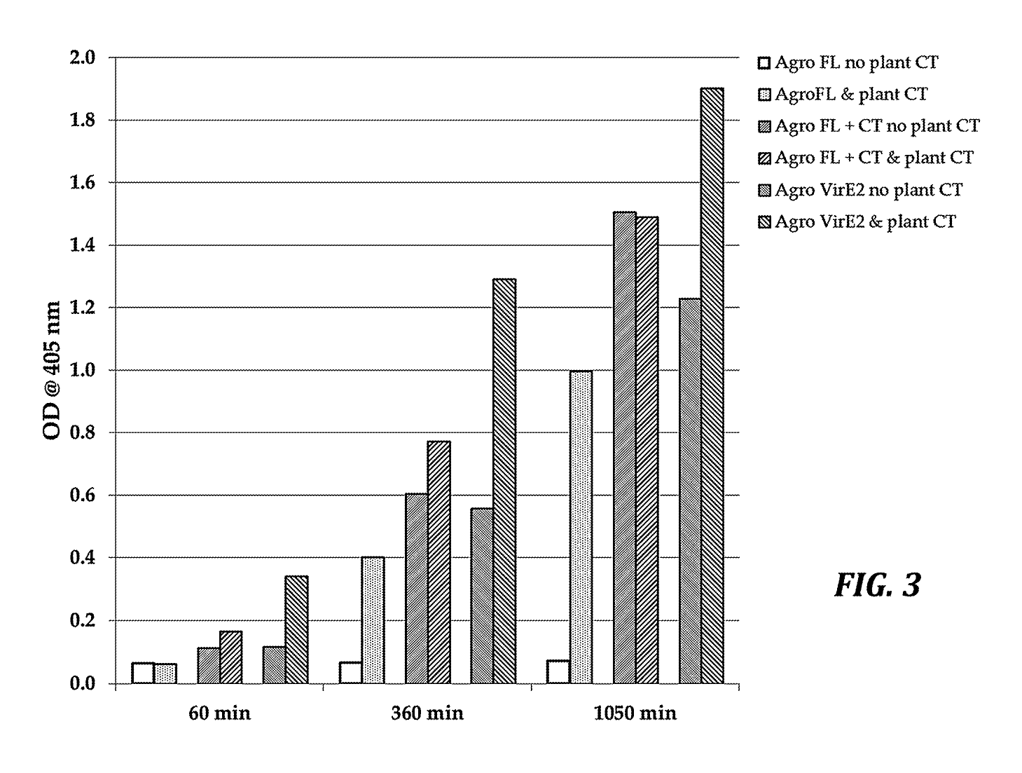

Specifically, a standard β-glucuronidase (GUS) reporter assay approach was used to ascertain the efficiency of GALLS- and VirE2-mediated plant transformation by Agrobacterium cells, either in the presence or absence of GALLS-CT. The results of the assays are set forth in FIG. 3, which illustrates the Optical Density at 405 nm, reflecting β-glucuronidase activity expressed in the indicated A. thaliana roots at six days post-exposure to the indicated Agrobacterium. After tissue collection, the GUS assays were conducted for 60, 360, and 1050 minutes.

One A. thaliana line was propagated for testing a transgenic line containing a heterologous gene encoding GALLS-CT under the control of the XVE promoter (Zuo, J. et al., Plant J. 24:265-273 (2000)), which is inducible by administration of estradiol (“plant CT”). Some plants were exposed to 5 μM estradiol to induce expression of GALLS-CT within the transgenic plants harboring the inducible GALLS-CT gene, whereas other plants were not treated with estradiol. After 24 hours of estradiol incubation (or cultivation without estradiol), the plant roots were harvested and infected with one of three modified A. tumefaciens, all of which harbor the β-glucuronidase reporter transgene in the T-DNA: 1) A. tumefaciens strain At1872, which lacks VirE2 expression, and is modified to express a mutant GALLS-FL gene from A. rhizogenes with a M8081 substitution to remove the internal start codon and, thus, prevents any alternate expression of the GALLS-CT protein (“Agro FL”); 2) A. tumefaciens strain At1872, which lacks VirE2, and is modified to express the wild-type GALLS-FL gene from A. rhizogenes, which permits expression of the GALLS-CT protein in addition to the GALLS-FL protein (“Agro FL+CT”) (see, e.g., Hodges, L. D., et al., “The Agrobacterium rhizogenes GALLS Gene Encodes Two Secreted Proteins Required for Genetic Transformation of Plants,” J. Bacteriol. 191(1):355-364 (2009), incorporated herein by reference in its entirety); and, 3) A. tumefaciens with an intact wild-type VirE2 pathway but no GALLS pathway (“Agro VirE2”). Thus, the plants, with or without internal GALLS-CT transgenic expression, were exposed to A. tumefaciens harboring the detectable transgene reporter in the T-DNA in three different transformation pathway contexts: GALLS-FL pathway (with or without GALLS-CT supplied by the A. tumefaciens) and wild-type VirE2. At six days post-exposure to the A. tumefaciens, the root segments were assayed for transient GUS expression by a spectrophotometric assay. Soluble proteins were extracted from root tissue and incubated with a substrate for the GUS enzyme (p-nitrophenyl β-D-glucuronide; PNPG) for 1, 6, and 17.5 hours (indicated in FIGS. 3 as 60, 360, and 1050 minutes, respectively). Upon cleavage by GUS, PNPG releases p-nitrophenol, which absorbs light at 405 nm. The GUS gene fusion system is described in more detail in Jefferson, R. A. and K. J. Wilson, Plant Mol. Biol. Manual B14:1-33 (1991), in S. B. Gelvin, R. A. Schilperoort, D. P. S. Verma, Eds., Kluwer Academic Publishers, Dordrecht, incorporated herein by reference in its entirety. For each data point, root segments from 10 plants were pooled, and data from a minimum of five pools were averaged.

Exemplary protocols are described below in Example 1, including steps for host Arabidopsis seed cultivation and root culture, Agrobacterium infection, assay for transient GUS activity, and tumorigenesis assay.

As demonstrated in FIG. 3, the presence of GALLS-CT resulted in the enhanced transient expression of GUS in the plant, indicating enhanced efficiency in GUS transgene transformation into the plant. This effect was observed for both GALLS- and VirE2-pathway mediated transformation systems. For transformation mediated by the GALLS-pathway, the effect occurred regardless of whether the GALLS-CT was provided by transgenic expression in the plant or by the infectious A. tumefaciens itself. Generally, the effect was greatest when the GALLS-CT was provided by both the A. tumefaciens and the host plant. Control assays demonstrated that estradiol incubation did not influence the above results other than by inducing GALLS-CT expression (not shown).

Accordingly, these data demonstrate that the presence of GALLS-CT, whether provided by the infecting bacterial microorganism or expressed transgenically within the host plant, enhances Agrobacterium-mediated transformation in plants.

### EXAMPLE 1

### Materials and Methods for Transformation of Arabidopsis

Seed Sterilization

1. Sterilize Arabidopsis seeds for 10 min in a solution of 50% bleach plus 0.1% SDS.

2. Rinse five times with sterile dH₂O.

3. Place seeds onto B5 medium plate (containing 50 μg/ml kanamycin or 10 μg/ml phosphinothricin or 20 μg/ml hygromycin B, whichever is appropriate). Include 100 μg/ml timentin in the medium to inhibit growth of any Agrobacterium that may be trapped under the seed coat.

4. Place plates at 4° C. for 2 days.

Root Culture

1. Germinate seeds in a growth chamber (23° C., 14 hr light, 10 hr dark) for 7-10 days.

2. Transfer seedling into a baby food jar containing B5 medium without antibiotics and grow for at least 10 days. Plants are ready for processing when the roots are long enough to get a minimum of 60 segments. Plants should be processed before a flower bolt emerges.

Agrobacterium Infection

1. Place Arabidopsis roots into a Petri dish, and add sterile dH₂O enough to wet.

2. Cut the roots from the plants and replace the plants back into baby food jars. Assign a code number to each plant. Cut the roots into 0.3-0.5 cm long segments.

3. Transfer bundles of root segments onto MS basal medium without antibiotics.

4. Place 2-3 drops of bacterial solution (grow Agrobacterium to a density of 10⁹ cells/ml [Klett=100] in YEP medium containing the appropriate antibiotics).

5. Wash the cells once in 0.9% NaCl, then resuspend the pellet in 0.9% NaCl at a concentration of 10⁸ cells/ml [Klett=10] to cover the root bundles and leave for 10 min.

Notes: Centrifugation of bacteria is done in a microfuge at top speed for 1 minute. For certain experiments, the bacteria may be resuspended at a Klett of 100, or a Klett of 1 or even less. All root segments must be infected within 30 minutes of being cut. Do not leave the segments for longer periods of time before infection.

6. Remove most of the bacterial solution, seal the Petri dishes with Parafilm, and co-culture the bacteria and root bundles for 40-50 hours in a growth chamber at 20° C.

7. 40-50 hours after co-cultivation, rinse the segments with dH₂O containing Timentin (100 μg/ml) or scrape off infected root segments on the surface of the medium. Transfer roots onto different types of medium according to the specific assay. For primary screening for mutants, separate roots into small bundles (up to 5 root segments/bundle). For secondary screening and quantitation, separate into individual root segments; do not use root bundles. A minimum of 60 root segments per plate is preferred.

8. Incubate the plates at 23° C., and score in 4-5 weeks.

9. When scoring for the phenotype of tumorigenesis, the percentage of root segments that give tumors is recorded and the morphology of the tumor (small yellow, large yellow, small green, or large green) should be indicated. The percentage of each morphology class should also be recorded.

Transient GUS Assay

1. Transfer root bundles onto Callus Inducing Medium (CIM) containing 100 μg/ml of Timentin, seal the plates with double layers of parafilm, and place in a growth chamber.

2. Take out the root segments, incubate with PNPG (a β-glucuronidase substrate), and measure the specific β-glucuronidase activity spectrophotometrically.

Tumorigenesis Assay

1. Transfer the roots onto MS basal medium with 100 μg/ml Timentin.

2. Seal the plates with double layers of parafilm and place them in a growth chamber for 4-5 weeks. Around 2 weeks after infection, you should be able to see small tumors appear.

Transformation to Kanamycin or Phosphinothricin Resistant Calli

1. Transfer the roots onto Callus Inducing Medium (CIM) containing 100 μg/ml of Timentin and either 50 μg/ml of kanamycin or 10 μg/ml of phosphinothricin.

2. Seal the plates with double layers of parafilm and place in a growth chamber for 4-5 weeks. Around 2 weeks after infection, small yellow calli should be visible.

Tissue Culture Media

1. MS Basal Medium (for 1 liter)
4.32 g MS minimal salts (Gibco)
0.5 g MES
1 ml Vitamin stock solution (1000×)
10 ml Myo-inositol stock solution (100×)
10 g Sucrose
Adjust pH to 5.7 with 1 N KOH
7.5 g Bacto Agar
Autoclave for 20-30 min

2. CIM (for 1 liter)
4.32 g MS minimal salts (Gibco)
0.5 MES
1 ml Vitamin stock solution (1000×)
10 ml Myo-inositol stock solution (100×)
20 g Glucose
1 ml IAA stock solution (1000×)
0.5 ml 2,4-D stock solution (2000×)
0.5 ml Kinetin stock solution (2000×)
Adjust pH to 5.7 with 1 N KOH
7.5 g Bacto Agar
Autoclave for no more than 20 min

3. B5 Medium
Gamborg's B5 medium (Gibco) (basal medium with minimum organics)
Dissolve the entire content from one bottle to make 1 liter medium
(If the B5 medium does not already contain sucrose, add 20 g sucrose)
Adjust pH to 5.7 with 1 N KOH
7.5 g Bacto Agar

4. Stock Solutions:

| | |
|---|---|
| Myo-inositol (100 X) | 10 mg/ml |
| Vitamin (1000 X) | 0.5 mg/ml Nicotinic Acid |
| | 0.5 mg/ml Pyridoxine |
| | 0.5 mg/ml Thiamine-HCl |
| IAA (1000 X) | 5 mg/ml in H2O (may use trace KOH) |
| 2,4-D (2000 X) | 1 mg/ml in H2O (may use trace KOH) |
| Kinetin (2000 X) | 0.6 mg/ml H2O (may use trace KOH) |

While illustrative embodiments have been illustrated and described, it will be appreciated that various changes can be made therein without departing from the spirit and scope of the invention.

1. A method of enhancing a single copy insertion of a first nucleic acid sequence into a plant cell genome comprising contacting the plant cell with the modified Agrobacterium cell that comprises the first nucleic acid sequence and a second nucleic acid sequence that encodes a GALLS-FL protein, wherein the first nucleic acid sequence is heterologous to the Agrobacterium cell and is operably linked to a first promoter sequence that facilitates expression of the first nucleic acid sequence in the plant cell.

2. The method of claim 1, wherein the second nucleic acid sequence that encodes the GALLS-FL protein is operably linked to a second promoter sequence to facilitate expression of the GALLS-FL protein in the Agrobacterium cell.

3. The method of claim 1, wherein the GALLS-FL protein comprises a first ATP-binding domain, a second ATP-binding domain, a helicase domain, a nuclear localization domain, and a GALLS-CT domain, wherein the GALLS-CT domain comprises at least two GALLS domains and a type-IV secretion signal.

4. The method of claim 1, wherein the GALLS-FL protein comprises an amino acid sequence with at least 70% identity to the amino acid sequence set forth in SEQ ID NO:2.

5. The method of claim 1, wherein the second nucleic acid sequence that encodes the GALLS-FL protein is derived from Agrobacterium rhizogenes.

6. The method of claim 1, wherein the second nucleic acid sequence that encodes the GALLS-FL protein is heterologous to the Agrobacterium cell.

7. The method of claim 1, wherein the modified Agrobacterium cell further comprises one or more nucleic acid sequences that encode one or more of VirA, VirG, VirB1-VirB11, VirD1, VirD2, VirD4, VirD5, VirC1, VirC2, and VirE3.

8. The method of claim 1, wherein the modified Agrobacterium cell does not express VirE2 polypeptide or VirE1 polypeptide.

9. The method of claim 8, wherein the modified Agrobacterium cell is an Agrobacterium rhizogenes, an Agrobacterium tumefaciens, or is derived therefrom.

10. The method of claim 1, wherein the first promoter sequence is an inducible promoter sequence.

11. The method of claim 1, wherein the first promoter sequence is a constitutive promoter in the plant cell nucleus.

12. The method of claim 1, wherein the first promoter sequence is a plant tissue-specific promoter.

13. The method of claim 1, wherein the first promoter sequence is homologous to a promoter sequence endogenous to the plant cell genome.

14. The method of claim 1, wherein the plant cell is selected from soybean, canola, corn, cotton, rice, alfalfa, wheat, potato, tomato, pepper, and the like.

15. The method of claim 1, wherein prior to the contacting step the first nucleic acid sequence is in a T-DNA domain, wherein the T-DNA domain is located on a plasmid or on a chromosome of the Agrobacterium cell.

16. The method of claim 15, wherein the T-DNA domain further comprises a third nucleic acid that encodes a selectable marker.

17. The method of claim 15, wherein the first nucleic acid sequence and the operably linked first promoter sequence are flanked on each side by one or more T-DNA border sequences.

18. The method of claim 17, wherein the first nucleic acid sequence and the operably linked first promoter sequence are further flanked on one side by an overdrive sequence.

19. The method of claim 15, wherein the plasmid is a Ti plasmid, an Ri plasmid, or a binary plasmid.

20. The method of claim 1, wherein the first nucleic acid sequence is heterologous to the plant cell genome.

21. The method of claim 1, further comprising propagating the plant cell.

22. The method of claim 1, further comprising inducing the expression of the first nucleic acid sequence in the plant cell or progeny thereof

23. The method of claim 1, wherein the single copy insertion rate enhanced by at least 20% over a reference method of plant transformation.

24. The method of claim 23, wherein the reference method comprises an Agrobacterium cell that expresses VirE2.

25. A method of transforming a plant cell with a first nucleic acid sequence, comprising contacting the plant cell with the modified Agrobacterium cell of claim 1.

26. A method of inducing plant susceptibility to Agrobacterium-mediated transformation, comprising providing GALLS-CT polypeptide in the cytosol of at least one cell of the plant.

27. A method of enhancing the efficiency of Agrobacterium-mediated transformation in a plant, comprising:
providing GALLS-CT polypeptide in the cytosol of at least one cell of the plant; and
contacting the plant with an Agrobacterium cell comprising a transgene capable of expression in the plant cell.

28. The method of claim 26 or claim 27, wherein providing GALLS-CT polypeptide in the cytosol comprises contacting the plant cell with an Agrobacterium cell that expresses GALLS-CT polypeptide.

29. The method of claim 26 or claim 27, wherein providing GALLS-CT polypeptide in the cytosol comprises providing for the expression of a heterologous nucleic acid that encodes GALLS-CT in the plant cell.

30. The method of claim 29, wherein the heterologous nucleic acid is stably integrated into the genome of the plant cell.

31. The method of claim 29, wherein the heterologous nucleic acid is transiently expressed in the plant cell.

32. The method of claim 26 or claim 27, wherein providing GALLS-CT polypeptide in the cytosol comprises contacting the plant cell with an Agrobacterium cell that expresses GALLS-CT polypeptide and providing for the expression of a heterologous nucleic acid that encodes GALLS-CT in the plant cell.

33. The method of claim 27, wherein GALLS-CT polypeptide is provided in the cytosol concurrently with or prior to contacting the plant with the Agrobacterium cell.

34. The method of claim 26 or claim 27, wherein the GALLS-CT polypeptide comprises at least two GALLS domains and a type-IV secretion domain.

35. The method of claim 34, wherein GALLS-CT protein is encoded by a nucleic acid derived from Agrobacterium rhizogenes.

36. The method of claim 34, wherein the GALLS-CT polypeptide has an amino acid sequence with at least 70% identity to the amino acid sequence set forth in SEQ ID NO:4.

37. The method of claim 26 or claim 27, wherein the Agrobacterium-mediated transformation is mediated by the Agrobacterium GALLS pathway or Agrobacterium VirE2 pathway.

38. The method of claim 26 or claim 27, wherein the plant is selected from soybean, canola, corn, cotton, rice, alfalfa, wheat, potato, tomato, pepper, and the like.

39. A transgenic plant, or component thereof, comprising a cell with a heterologous nucleic acid sequence encoding GALLS-CT operably linked to a promoter sequence.

40. The transgenic plant, or component thereof, of claim 39, wherein the heterologous nucleic acid sequence is stably integrated into the genome of the cell.


SEQUENCE LISTING (18 sequences)

SEQ ID NO: 1 - DNA, 5310 bp, Agrobacterium rhizogenes
atgccaaccgacgacattgtaatgtccgatcccggaatggctgctgttgacacgtctgtccctatgcgcttccagacagatcttcgccagttcagtaacctgctggacgagagcaatattgtgggatgggtcgtaaaccacgccgcaaatcgcaatgcgtgcttcggacaagaccaacttaagatttcggcggctcgggcactcacgcactacaaacggccggtacaagaaatcgacctgctattccaacaggtcgtcgcaagcgacaaactgacccggcttgattctccagatggccaacctctctacacctctacagagctgagagctgcggaacagaatactgtacgtcatgtcactaatctttccgcagaagaaagcttcggcgttccgggaaccattgttgatatggtcgccgatagaagcattttcacaccggagctcagaggagcattacattatctgtctaggcgaaatcgagcttccactatggtgggcgtagcgggttccgcaaaaacaagtgtacttggagccctgaacgaagcggtggacaggttcaacgaggaagcgctcccgaccgagaaaataaagttgattgccttcgtacccaccaaccgggcggcccaggagcttcgtgagaaaggtcttcgcgacgtcgcgacgactttcaaggcgaaagaccgcaccatcggaaaaaataccatcgtcgtgatcgacgaaatgtcgatggccaagacgcaggagatcgcgcaactaatcgagacagttgcaaatgccacttcctatcacccaaccgaaaggccgaaactgatatgtgtgggagacgatcggcaactgccccctgttggacctggtgatctgcttccgattattatggagcaagccggctgctatgaactcgtccagccgcttcgacagctcgacgcgcggtcgcgaatagaaacagaaaagctcggacgtgacatacgacgcgatgccaagagcgccgtcgggcactatctcacagcgatcgaacaaatgggcctcgtccatttcgtatatgctcctcctggcggctcaaacgtcagggcgtcagaccagatcactgaccgcatgctggaggagatgaatagagtttgcgaccgatatccggtggatgatcgtcttgtgatggcacactccaacaggagcgttaatcgcgccaatatcgctcttcatcagaggtttgttgacaaatccggattgcaagaccagcaaatgagtgtgtggtcaagcgtaatagatagtggaagttcggaatctggaatagatgagggttcgtcgagacgcgttcagcaaaggattaatctcgtcttgggagacaggattatattcagcgaaaaccgtctgcaagcggacatccgcaatggtacttttgcaactgtagtcggacttaatagcacagaaccttcccgagctgagaatggcacatctgctggggttcgcattactgcaaggctagacggatctggacgaaccgtcacttggaccgatagcgagtttaagggttttacctacggctatgctgctacaatttataagagtcagggggccacagttgatcacgccattctcctctgtgatggtgctctttcagataagctgacctatgtcggcctgacgaggcatcgcagtcgtctcgacgtcattgcttctcccctcgtggcaaacaatgttgaggctctcagtgcaaagctcactcagcaaagcgcgccaaataattccattcaattcccggcgctcgaacctgctgccttccaagagcgctcgtcggtttcgaacgcgcacttgctaaggccgctcgaccagatggaaatctctccaacgacgccgctttccttccaaggcagacaagaaagctccttcggggagaaccagctgcacgaaccatccacatcggatgtggaaatgccggaaagcgcggacctttacccgagcgccattgacttcgaagttttccagtcttctgtcatcacgactgaagagcaaatgcaaccggaattctgcgggtcggaatcgacggctaacacctcagcccacgaaagccaccaagtcacgatcgcaacggtaacaaacctggggatagcgatcgagaaacggaaacgagcggcggcaaaggaagaaatcgattctcgcaaaaaaatggcccgtcactcagttagttctgccgaggaagttgaagatatcacttccctattcgaacgattgaaactggcaacctcggtcacgaacacagttcaaccgacgtatgcggacgtcgatcatatcatcggtactgatggccgagtgggtccaataccgcaccgggaagatgatccgggatcaaattgggaattgtcgtcaacgaacgccccctcaaacgtctcagagaaacgatcgaccaaagactcaggagaaaaaaatatggcttcgaatgaccagaaaatgcaaggcgctgactctgcaggcagcgagcgtcatttttcgcagacttccattgtggaagccctgcaggctggcgatcacaagaagctcgtggaaacgctcgccgcaaccggctcccttgtcgagaaggcggacgccgggtcggcgctgaaagcaatggcggatgcttacgaaagggatatcaaggcagatcccgaaaaggtccgcatgctctatgccaatcggcgcgttgatgtagatgcgctggccaaggagatcgaaaaacagggccttgcatccggtcgcctgagtggcgaccctgtcagcttcaaggcgttcaacaacaaggatatccgtttcagggtcggcgacaggctcagcgtcaccagcagctacaggaatctcattgccggtgaagccggccggatcgaaaagatcgaaggcaaatccgttaccttcaaacccgatggcagcagtcagagcaagaccttcgaggccagcaccggcgagccgggtgacaggggcgtcaagggctacaaaatcgaaggcccgatgacggtcatcggctccgcctatgaacttacgcgacatgatgacggcaaacgtgttgaccaaatctatctcttgaacgacgctgcccatggcatcaacgccaatggcgccttgctgagcaaggccgacgagcggacgcagcttttcacttcgctcaaggacaccgccgacagagacatcttggccaggcaactgggaaagccgggcagcgagctctaccaggacaaattcccagccttgccggactacacagccgccatcgatcggattgcaaaacgccaggaagttgtcgcggcacgtggcgaggagaaaacttccaccccgatagatctggcgccccaaacgccgcccgacgctgccgatgcggcagtcaataaccagaaaagaatcgtcgaggccttgcaggcaggcgaccacaagaagcttgtggaaacgctagccgcaaccggctcccttgttgagaacacaaatctcgtatcaacgttgaaagcgatagcgcagacttatgaaagagacattagtgcggaccccgagaaagtccgcgctctctacgccaacaaacgtgatcatgtagatttgcttgccgaagagatcgaaaagcagggccttgcatccggtcgcctgagtggcgaccctgtcagcttcaaggcgtttaacaacaaggatatccgtttcagggtcggtgacaggctcagcgtcaccagcagctataggaatctcattgccggtgaagccggccggatcgaaaagatcgaaggcaaatccgttaccttcaaacccgatggcagcagtcagagcaagaccttcgaggccagcaccggcgagccgggtgacaggggtgtcaagggctataagattgaggggcccacgacaatcatcggctccgcctacgaaatcagaaatcacgacgatggcaggcgcgtcgatcaggtttatgtcttgcacagtgccattttcggctcaaatgccgcgagcgccctgttgagcaaggccgacgaacgaacccaggttttcaccgtgcaaagcgacacggctgacagacacatcttggccagccaactaagcaagcctggcagcgagctctatcaggacaaattcccagtctcgccggactacgcagccgcgatcgaccggatcgcgaaacgcgaggaacttagggccggacgtggcgaggaaagtcctactgttcaaatggagccggtgaaccaaacgccctacgaggtgggacattggacgcctgacagtcaaaaatctgtggtggaagccctgcaggctggcgatcacaagaagctcgtggaaacgctcgccgcaaccggctcccttgtcgagaaggcggacgccgggtcggcgctgaaagcaatggcggatgcttacgaaagggatatcaaggcagatcccgaaaaggtccgcatgctctatgccaatcggcgcgttgatgtagatgcgctggccaaggagatcgaaaaacagggccttgcatccggtcgcctgagtggcgaccctgtcagcttcaaggcgttcaacaacaaggatatccgtttcagggtcggcgacaggctcagcgtcaccagcagctacaggaatctcattgccggtgaagccggccggatcgaaaagatcgaaggcaaatccgttaccttcaaacccgatggcagcagtcagagcaagaccttcgaggccagcaccggcgagccgggtgacaggggcgtcaagggctacaaaatcgaaggcccgatgacggtcatcggctccgcctatgaacttacgcgacatgatgacggcaaacgtgttgaccaaatctatctcttgaacgacgctgcccatggcatcaacgccaatggcgccttgctgagcaaggccgacgagcggacgcagcttttcacttcgctcaaggacaccgccgacagagacatcttggccaggcaactgggaaagccgggcagcgagctctaccaggacaaattcccagccttgccggactacacagccgccatcgatcggattgcaaaacgccaggaagttgtcgcggcacgtggcgagatcaagccgattgagcgcaccgaagcgcccgaaataccccaggttcaagctactatcgttggcgagacctcgatcattcggttgcccgatcggataaaaaccaatcgcgagaagccccaagaacttgatgtagtctcgaaagaagggcacgaaactacaggtagtctgaggcagagtgtccaagagagtaatgcagcaccaaaacagacttcgccgaaggcggcaaatgacgtggatcggctgacacgggattttgacgagcgcatccgcgtccgcggggatggacgtggactctaa

SEQ ID NO: 2 - protein, 1769 aa, Agrobacterium rhizogenes
MPTDDIVMSDPGMAAVDTSVPMRFQTDLRQFSNLLDESNIVGWVVNHAANRNACFGQDQLKISAARALTHYKRPVQEIDLLFQQVVASDKLTRLDSPDGQPLYTSTELRAAEQNTVRHVTNLSAEESFGVPGTIVDMVADRSIFTPELRGALHYLSRRNRASTMVGVAGSAKTSVLGALNEAVDRFNEEALPTEKIKLIAFVPTNRAAQELREKGLRDVATTFKAKDRTIGKNTIVVIDEMSMAKTQEIAQLIETVANATSYHPTERPKLICVGDDRQLPPVGPGDLLPIIMEQAGCYELVQPLRQLDARSRIETEKLGRDIRRDAKSAVGHYLTAIEQMGLVHFVYAPPGGSNVRASDQITDRMLEEMNRVCDRYPVDDRLVMAHSNRSVNRANIALHQRFVDKSGLQDQQMSVWSSVIDSGSSESGIDEGSSRRVQQRINLVLGDRIIFSENRLQADIRNGTFATVVGLNSTEPSRAENGTSAGVRITARLDGSGRTVTWTDSEFKGFTYGYAATIYKSQGATVDHAILLCDGALSDKLTYVGLTRHRSRLDVIASPLVANNVEALSAKLTQQSAPNNSIQFPALEPAAFQERSSVSNAHLLRPLDQMEISPTTPLSFQGRQESSFGENQLHEPSTSDVEMPESADLYPSAIDFEVFQSSVITTEEQMQPEFCGSESTANTSAHESHQVTIATVTNLGIAIEKRKRAAAKEEIDSRKKMARHSVSSAEEVEDITSLFERLKLATSVTNTVQPTYADVDHIIGTDGRVGPIPHREDDPGSNWELSSTNAPSNVSEKRSTKDSGEKNMASNDQKMQGADSAGSERHFSQTSIVEALQAGDHKKLVETLAATGSLVEKADAGSALKAMADAYERDIKADPEKVRMLYANRRVDVDALAKEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPMTVIGSAYELTRHDDGKRVDQIYLLNDAAHGINANGALLSKADERTQLFTSLKDTADRDILARQLGKPGSELYQDKFPALPDYTAAIDRIAKRQEVVAARGEEKTSTPIDLAPQTPPDAADAAVNNQKRIVEALQAGDHKKLVETLAATGSLVENTNLVSTLKAIAQTYERDISADPEKVRALYANKRDHVDLLAEEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPTTIIGSAYEIRNHDDGRRVDQVYVLHSAIFGSNAASALLSKADERTQVFTVQSDTADRHILASQLSKPGSELYQDKFPVSPDYAAAIDRIAKREELRAGRGEESPTVQMEPVNQTPYEVGHWTPDSQKSVVEALQAGDHKKLVETLAATGSLVEKADAGSALKAMADAYERDIKADPEKVRMLYANRRVDVDALAKEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPMTVIGSAYELTRHDDGKRVDQIYLLNDAAHGINANGALLSKADERTQLFTSLKDTADRDILARQLGKPGSELYQDKFPALPDYTAAIDRIAKRQEVVAARGEIKPIERTEAPEIPQVQATIVGETSIIRLPDRIKTNREKPQELDVVSKEGHETTGSLRQSVQESNAAPKQTSPKAANDVDRLTRDFDERIRVRGDGRGL

SEQ ID NO: 3 - DNA, 2889 bp, Agrobacterium rhizogenes
atggcttcgaatgaccagaaaatgcaaggcgctgactctgcaggcagcgagcgtcatttttcgcagacttccattgtggaagccctgcaggctggcgatcacaagaagctcgtggaaacgctcgccgcaaccggctcccttgtcgagaaggcggacgccgggtcggcgctgaaagcaatggcggatgcttacgaaagggatatcaaggcagatcccgaaaaggtccgcatgctctatgccaatcggcgcgttgatgtagatgcgctggccaaggagatcgaaaaacagggccttgcatccggtcgcctgagtggcgaccctgtcagcttcaaggcgttcaacaacaaggatatccgtttcagggtcggcgacaggctcagcgtcaccagcagctacaggaatctcattgccggtgaagccggccggatcgaaaagatcgaaggcaaatccgttaccttcaaacccgatggcagcagtcagagcaagaccttcgaggccagcaccggcgagccgggtgacaggggcgtcaagggctacaaaatcgaaggcccgatgacggtcatcggctccgcctatgaacttacgcgacatgatgacggcaaacgtgttgaccaaatctatctcttgaacgacgctgcccatggcatcaacgccaatggcgccttgctgagcaaggccgacgagcggacgcagcttttcacttcgctcaaggacaccgccgacagagacatcttggccaggcaactgggaaagccgggcagcgagctctaccaggacaaattcccagccttgccggactacacagccgccatcgatcggattgcaaaacgccaggaagttgtcgcggcacgtggcgaggagaaaacttccaccccgatagatctggcgccccaaacgccgcccgacgctgccgatgcggcagtcaataaccagaaaagaatcgtcgaggccttgcaggcaggcgaccacaagaagcttgtggaaacgctagccgcaaccggctcccttgttgagaacacaaatctcgtatcaacgttgaaagcgatagcgcagacttatgaaagagacattagtgcggaccccgagaaagtccgcgctctctacgccaacaaacgtgatcatgtagatttgcttgccgaagagatcgaaaagcagggccttgcatccggtcgcctgagtggcgaccctgtcagcttcaaggcgtttaacaacaaggatatccgtttcagggtcggtgacaggctcagcgtcaccagcagctataggaatctcattgccggtgaagccggccggatcgaaaagatcgaaggcaaatccgttaccttcaaacccgatggcagcagtcagagcaagaccttcgaggccagcaccggcgagccgggtgacaggggtgtcaagggctataagattgaggggcccacgacaatcatcggctccgcctacgaaatcagaaatcacgacgatggcaggcgcgtcgatcaggtttatgtcttgcacagtgccattttcggctcaaatgccgcgagcgccctgttgagcaaggccgacgaacgaacccaggttttcaccgtgcaaagcgacacggctgacagacacatcttggccagccaactaagcaagcctggcagcgagctctatcaggacaaattcccagtctcgccggactacgcagccgcgatcgaccggatcgcgaaacgcgaggaacttagggccggacgtggcgaggaaagtcctactgttcaaatggagccggtgaaccaaacgccctacgaggtgggacattggacgcctgacagtcaaaaatctgtggtggaagccctgcaggctggcgatcacaagaagctcgtggaaacgctcgccgcaaccggctcccttgtcgagaaggcggacgccgggtcggcgctgaaagcaatggcggatgcttacgaaagggatatcaaggcagatcccgaaaaggtccgcatgctctatgccaatcggcgcgttgatgtagatgcgctggccaaggagatcgaaaaacagggccttgcatccggtcgcctgagtggcgaccctgtcagcttcaaggcgttcaacaacaaggatatccgtttcagggtcggcgacaggctcagcgtcaccagcagctacaggaatctcattgccggtgaagccggccggatcgaaaagatcgaaggcaaatccgttaccttcaaacccgatggcagcagtcagagcaagaccttcgaggccagcaccggcgagccgggtgacaggggcgtcaagggctacaaaatcgaaggcccgatgacggtcatcggctccgcctatgaacttacgcgacatgatgacggcaaacgtgttgaccaaatctatctcttgaacgacgctgcccatggcatcaacgccaatggcgccttgctgagcaaggccgacgagcggacgcagcttttcacttcgctcaaggacaccgccgacagagacatcttggccaggcaactgggaaagccgggcagcgagctctaccaggacaaattcccagccttgccggactacacagccgccatcgatcggattgcaaaacgccaggaagttgtcgcggcacgtggcgagatcaagccgattgagcgcaccgaagcgcccgaaataccccaggttcaagctactatcgttggcgagacctcgatcattcggttgcccgatcggataaaaaccaatcgcgagaagccccaagaacttgatgtagtctcgaaagaagggcacgaaactacaggtagtctgaggcagagtgtccaagagagtaatgcagcaccaaaacagacttcgccgaaggcggcaaatgacgtggatcggctgacacgggattttgacgagcgcatccgcgtccgcggggatggacgtggactctaa

SEQ ID NO: 4 - protein, 962 aa, Agrobacterium rhizogenes
MASNDQKMQGADSAGSERHFSQTSIVEALQAGDHKKLVETLAATGSLVEKADAGSALKAMADAYERDIKADPEKVRMLYANRRVDVDALAKEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPMTVIGSAYELTRHDDGKRVDQIYLLNDAAHGINANGALLSKADERTQLFTSLKDTADRDILARQLGKPGSELYQDKFPALPDYTAAIDRIAKRQEVVAARGEEKTSTPIDLAPQTPPDAADAAVNNQKRIVEALQAGDHKKLVETLAATGSLVENTNLVSTLKAIAQTYERDISADPEKVRALYANKRDHVDLLAEEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPTTIIGSAYEIRNHDDGRRVDQVYVLHSAIFGSNAASALLSKADERTQVFTVQSDTADRHILASQLSKPGSELYQDKFPVSPDYAAAIDRIAKREELRAGRGEESPTVQMEPVNQTPYEVGHWTPDSQKSVVEALQAGDHKKLVETLAATGSLVEKADAGSALKAMADAYERDIKADPEKVRMLYANRRVDVDALAKEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPMTVIGSAYELTRHDDGKRVDQIYLLNDAAHGINANGALLSKADERTQLFTSLKDTADRDILARQLGKPGSELYQDKFPALPDYTAAIDRIAKRQEVVAARGEIKPIERTEAPEIPQVQATIVGETSIIRLPDRIKTNREKPQELDVVSKEGHETTGSLRQSVQESNAAPKQTSPKAANDVDRLTRDFDERIRVRGDGRGL

SEQ ID NO: 5 - protein, 14 aa, Agrobacterium rhizogenes
RASTMVGVAGSAKT

SEQ ID NO: 6 - protein, 13 aa, Agrobacterium rhizogenes
RTIGKNTIVVIDE

SEQ ID NO: 7 - protein, 20 aa, Agrobacterium rhizogenes
KLICVGDDRQLPPVGPGDLL

SEQ ID NO: 8 - protein, 10 aa, Agrobacterium rhizogenes
DDRLVMAHSN

SEQ ID NO: 9 - protein, 9 aa, Agrobacterium rhizogenes
GDRIIFSEN

SEQ ID NO: 10 - protein, 9 aa, Agrobacterium rhizogenes
RLDGSGRTV

SEQ ID NO: 11 - protein, 15 aa, Agrobacterium rhizogenes
GYAATIYKSQGATVD

SEQ ID NO: 12 - protein, 15 aa, Agrobacterium rhizogenes
LTYVGLTRHRSRLDV

SEQ ID NO: 13 - protein, 20 aa, Agrobacterium rhizogenes
KRKRAAAKEEIDSRKKMARH

SEQ ID NO: 14 - protein, 366 aa, Agrobacterium rhizogenes
SAEEVEDITSLFERLKLATSVTNTVQPTYADVDHIIGTDGRVGPIPHREDDPGSNWELSSTNAPSNVSEKRSTKDSGEKNMASNDQKMQGADSAGSERHFSQTSIVEALQAGDHKKLVETLAATGSLVEKADAGSALKAMADAYERDIKADPEKVRMLYANRRVDVDALAKEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPMTVIGSAYELTRHDDGKRVDQIYLLNDAAHGINANGALLSKADERTQLFTSLKDTADRDILARQLGKPGSELYQDKFPALPDYTAAIDRIAKRQEVVAARGE

SEQ ID NO: 15 - protein, 266 aa, Agrobacterium rhizogenes
NQKRIVEALQAGDHKKLVETLAATGSLVENTNLVSTLKAIAQTYERDISADPEKVRALYANKRDHVDLLAEEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPTTIIGSAYEIRNHDDGRRVDQVYVLHSAIFGSNAASALLSKADERTQVFTVQSDTADRHILASQLSKPGSELYQDKFPVSPDYAAAIDRIAKREELRAGRGE

SEQ ID NO: 16 - protein, 266 aa, Agrobacterium rhizogenes
SQKSVVEALQAGDHKKLVETLAATGSLVEKADAGSALKAMADAYERDIKADPEKVRMLYANRRVDVDALAKEIEKQGLASGRLSGDPVSFKAFNNKDIRFRVGDRLSVTSSYRNLIAGEAGRIEKIEGKSVTFKPDGSSQSKTFEASTGEPGDRGVKGYKIEGPMTVIGSAYELTRHDDGKRVDQIYLLNDAAHGINANGALLSKADERTQLFTSLKDTADRDILARQLGKPGSELYQDKFPALPDYTAAIDRIAKRQEVVAARGE

SEQ ID NO: 17 - protein, 15 aa, Agrobacterium rhizogenes
  misc_feature (2)..(8): Xaa can be any naturally occurring amino acid
RXXXXXXXRXRXRXX

SEQ ID NO: 18 - protein, 13 aa, Agrobacterium rhizogenes
RLTRDFDERIRVR